SEQUENCE LISTING (31 sequences)

SEQ ID NO: 1 - DNA, 1827 bp, Pueraria montana
  Pueraria montana var. lobata kudzu vine isoprene synthase (IspS)
atggcaaccaaccttttatgcttgtctaataaattatcgtcccccacaccaacaccaagtactagatttccacaaagtaagaacttcatcacacaaaaaacatctcttgccaatcccaaaccttggcgagttatttgtgctacgagctctcaatttacccaaataacagaacataatagtcggcgttcagctaattaccagccaaacctctggaattttgaatttctgcagtctctggaaaatgaccttaaggtggaaaaactagaagagaaggcaacaaagctagaggaggaggtacgatgcatgatcaacagagtagacacacaaccattaagcttactagaattgatcgacgatgtccagcgtctaggattgacctacaagtttgagaaggacataatcaaagcccttgagaatattgttttgctggatgagaataagaaaaataaaagtgacctccatgctactgctctcagcttccgtttacttagacaacatggctttgaggtttcccaagatgtgtttgagagatttaaggacaaggagggaggtttcagtggtgaacttaaaggtgatgtgcaagggttgctgagtctatatgaagcatcctatcttggctttgagggagaaaatctcttggaggaggcaaggacattttcaataacacatctcaagaacaacctaaaagaaggaataaacaccaaagtggcagaacaagttagtcatgcactggaacttccctatcatcaaagattgcatagactagaagcacgatggttccttgacaaatatgaaccaaaggaaccccaccatcagttactactcgagcttgcaaagctagatttcaatatggtgcaaacattgcaccagaaagaactgcaagacctgtcaaggtggtggacggagatggggctagcaagcaagctagactttgtccgagacagattaatggaagtgtatttttgggcgttgggaatggcacctgatcctcaattcggtgaatgtcgtaaagctgtcactaaaatgtttggattggtcaccatcatcgatgatgtatatgacgtttatggtactttggatgagctacaactcttcactgatgctgttgagagatgggacgtgaatgccataaacacacttccagactacatgaagttgtgcttcctagcactttataacaccgtcaatgacacgtcttatagcatccttaaagaaaaaggacacaacaacctttcctatttgacaaaatcttggcgtgagttatgcaaagcattccttcaagaagcaaaatggtcgaacaacaaaatcattccagcatttagcaagtacctggaaaatgcatcggtgtcctcctccggtgtggctttgcttgctccttcctacttctcagtgtgccaacaacaagaagatatctcagaccatgctcttcgttctttaactgatttccatggccttgtgcgctcctcatgcgtcattttcagactctgcaatgatttggctacctcagcggctgagctagagaggggtgagacgacaaattcaataatatcttatatgcatgagaatgacggcacttctgaagagcaagcacgtgaggagttgagaaaattgatcgatgcagagtggaagaagatgaaccgagagcgagtttcagattctacactactcccaaaagcttttatggaaatagctgttaacatggctcgagtttcgcattgcacataccaatatggagacggacttggaaggccagactacgccacagagaatagaatcaagttgctacttatagacccctttccaatcaatcaactaatgtacgtgtaa

SEQ ID NO: 2 - protein, 608 aa, Pueraria montana
  Pueraria montana var. lobata kudzu vine isoprene synthase (IspS)
MATNLLCLSNKLSSPTPTPSTRFPQSKNFITQKTSLANPKPWRVICATSSQFTQITEHNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 3 - DNA, 1707 bp, Artificial Sequence
  Description of Artificial Sequencesynthetic Pueraria montana var. lobata kudzu vine isoprene synthase (IspS) cDNA codon-adjusted for expression in yeast
catatgatctgtgctacatcatctcagtttacccaaattaccgaacacaattccagaagaagtgctaattatcaaccgaacctatggaattttgaatttttacaatcactggagaacgatttaaaagttgaaaagttagaggaaaaggctaccaaacttgaggaagaagttagatgtatgattaatagggttgacactcaacctttatcactattggaattaattgatgatgtacaacgtctgggtttgacctataaattcgaaaaagacataataaaggctctagaaaatatagtcttgttagacgaaaacaaaaagaataaaagcgacctacatgcgacagcattatcttttagacttttaagacaacatggctttgaagtatcccaagatgtattcgaaagatttaaagataaagagggaggttttagcggtgaattaaaaggtgatgtgcaaggattattgagtttgtatgaagcctcctatttaggattcgagggtgagaacctattagaagaagctcgtacattttccattactcatttgaaaaacaatttaaaggaaggtataaacaccaaagttgctgaacaagtttcgcatgcgctggaattaccatatcaccagagactacataggcttgaagctagatggttccttgataagtacgaacctaaggagccacatcaccaattgttactagaattagccaaactggatttcaatatggtacagacccttcatcaaaaagagttgcaagatttgtcaagatggtggaccgagatgggcctagccagtaaattggattttgttagagatagattaatggaagtttatttctgggcattaggtatggcaccagatccacagtttggggaatgtcgtaaagctgtcacaaaaatgtttggtttggttacaatcatagacgacgtctatgatgtatatggtactctagacgagttgcaattattcactgatgctgttgaaagatgggacgttaatgcaattaatactctacctgattatatgaagttgtgttttctagcattgtataatacggtaaacgatacatcatattctattttgaaagaaaagggtcataacaatctttcttacttgacaaagtcctggagagaactatgtaaggccttcctacaagaggcaaaatggtccaataataaaattataccagcattttcgaaatacttagagaatgcaagcgtatcctcaagtggtgttgcgttgttagctccaagttattttagtgtttgtcaacaacaagaagacatctccgaccatgcacttcgttcattgacggactttcatgggcttgtgagatcatcctgcgtgatcttcaggctttgcaacgatttagctacctctgctgctgaattagaacgtggtgagacgaccaatagcattatatcttacatgcatgagaatgacgggacatccgaggagcaggcaagagaagaattacgtaagttgatcgacgccgagtggaaaaagatgaatcgtgaaagagtcagcgactccacactgcttccaaaagccttcatggagatcgctgttaacatggccagagttagtcattgtacttatcagtatggcgatggattgggtagaccggattatgcaactgagaatagaatcaaacttttattgatcgatccgtttcccattaaccagctaatgtatgtataatctaga

SEQ ID NO: 4 - DNA, 1707 bp, Artificial Sequence
  Description of Artificial Sequencesynthetic Pueraria montana var. lobata kudzu vine isoprene synthase (IspS) cDNA codon-adjusted for expression in Neurospora crassa
catatgatttgtgccacctctagccagtttactcagattaccgagcataactctcgtcggtccgccaactaccagccgaacctgtggaacttcgagtttctccaaagcctcgagaacgaccttaaagtcgagaagctggaggaaaaggccaccaagttggaagaagaggttcggtgtatgatcaaccgcgtcgatacacagcccctgtctctgctcgagctcatcgacgacgtccaacgcctcggccttacgtacaagttcgaaaaggatatcatcaaggcactggagaacatcgtgcttctcgacgaaaacaagaagaacaaatctgacctgcacgcaaccgccttgtccttccgcttgttgcgccagcacggtttcgaggtgtcgcaggacgtgtttgagcgcttcaaggacaaggagggtggtttctctggcgagctgaagggagatgttcaaggccttttgagcctctacgaggccagctacctcggtttcgaaggagagaacctccttgaagaagctcgtacgttctccatcacccatctcaagaacaaccttaaggaaggtatcaacacgaaggtggcagagcaagtttcccacgctctcgagctgccttaccaccagcgcctgcatcgcttggaggcccgctggttcctggataagtacgaaccaaaggagccccaccatcagctgctcctcgagctggctaaacttgacttcaatatggttcagactctgcaccaaaaggaactgcaagacctctcgcgttggtggaccgaaatgggtcttgcaagtaagctggactttgtgcgtgaccgccttatggaggtgtacttctgggctctgggcatggctccagacccccagttcggtgagtgccgcaaggctgtgacgaagatgtttggtctggttaccattattgacgacgtgtacgacgtttatggaacgctcgacgagcttcagcttttcacagacgccgtggagcggtgggacgttaatgccatcaacaccctgcccgattacatgaagctgtgctttttggccctttacaacacagtgaatgatacgtcgtactctattctcaaagagaagggtcacaacaacctctcttacttgactaagtcgtggcgtgaactgtgcaaggccttccttcaggaggcgaaatggagcaataataagatcattcccgccttctccaagtatctggaaaacgccagtgtgtccagctcgggcgtcgccttgctggcccccagttatttctctgtttgccagcaacaggaggatatctccgaccatgccctgcgttccctgaccgatttccatggactggtccgctcctcctgcgttattttccgtctgtgtaacgatctggccactagcgctgcggagctggaacgcggagagacgaccaactccattatctcctacatgcacgagaacgatggcaccagtgaagagcaggcccgcgaggagcttcgcaaactgatcgacgctgagtggaagaagatgaatcgtgagcgggttagcgattcgaccctcctgcccaaggcttttatggaaattgccgttaacatggctcgcgtgtcccattgtacctaccagtacggcgacggtttgggccgtccggattacgcaaccgagaaccgcatcaagctccttctgatcgacccgttcccgatcaaccagctcatgtatgtctaatctaga

SEQ ID NO: 5 - protein, 564 aa, Artificial Sequence
  Description of Artificial Sequencesynthetic Pueraria montana var. lobata kudzu vine isoprene synthase (IspS) lacking N-terminus chloroplast transit peptide (cpTP) expressed in yeast or Neurospora crassa
ICATSSQFTQITEHNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 6 - DNA, 1788 bp, Populus alba
  white poplar isoprene synthase (IspS)
atggcaactgaattattgtgcttgcaccgtccaatctcactgacacacaaacttttcagaaatcccttacctaaagtcatccaggccactcccttaactttgaaactcagatgttctgtaagcacagaaaacgtcagcttcacagaaacagaaacagaagccagacggtctgccaattatgaaccaaatagctgggattatgattatttgctgtcttcagacactgacgaatcgattgaggtatacaaagacaaggccaaaaagctggaggctgaggtgagaagagagattaacaatgaaaaggcagagtttttgactctgcttgaactgatagataatgtccaaaggttaggattgggttaccggttcgagagtgacataaggggagcccttgatagatttgtttcttcaggaggatttgatgctgttacaaaaactagccttcatggtactgctcttagcttcaggcttctcagacagcatggttttgaggtctctcaagaagcgttcagtggattcaaggatcaaaatggcaatttcttggaaaaccttaaggaggacatcaaggcaatactaagcctatatgaagcttcatttcttgcattagaaggagaaaatatcttggatgaggccaaggtgtttgcaatatcacatctaaaagagctcagcgaagaaaagattggaaaagagctggccgaacaggtgaatcatgcattggagcttccattgcatcgcaggacgcaaagactagaagctgtttggagcattgaagcataccgtaaaaaggaagatgcaaatcaagtactgctagaacttgctatattggactacaacatgattcaatcagtataccaaagagatcttcgcgagacatcaaggtggtggaggcgagtgggtcttgcaacaaagttgcattttgctagagacaggttaattgaaagcttttactgggcagttggagttgcgttcgagcctcaatacagtgattgccgtaattcagtagcaaaaatgttttcatttgtaacaatcattgatgatatctatgatgtttatggtactctggacgagttggagctatttacagatgctgttgagagatgggatgttaatgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggttgtacaataagtccacaccaacatttgatgactatttcggaaatgcatggaaatcatcctcagggcctcttcaactagtttttgcctactttgccgtggttcaaaacatcaagaaagaggaaattgaaaacttacaaaagtatcatgataccatcagtaggccttcccacatctttcgtctttgcaacgacctggcttcagcatcggctgagatagcgagaggtgaaacagcgaattctgtatcatgctacatgcgtacaaaaggcatttctgaggagcttgctactgaatccgtaatgaacttgatcgacgaaacctggaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataa

SEQ ID NO: 7 - protein, 595 aa, Populus alba
  white poplar isoprene synthase (IspS)
MATELLCLHRPISLTHKLFRNPLPKVIQATPLTLKLRCSVSTENVSFTETETEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 8 - DNA, 1751 bp, Artificial Sequence
  Description of Artificial SequenceSc-k-IspS alternate version 1 of Pueraria montana var. lobata kudzu vine isoprene synthase (IspS) codon-adjusted for expression in yeast
gcgatcgcacacacataaataaacaaaatgatctgtgcaacttcttcccaattcacccaaataaccgaacataattcccgtagaagtgcaaattaccaaccaaatttgtggaattttgaatttcttcaatccttggagaacgatttgaaagtggaaaagttggaggaaaaagctaccaagcttgaagaagaagtgagatgtatgataaacagagtggatacccagccattatctttgttagaactaatcgatgacgttcaaagactaggtctaacatataagtttgagaaggatattattaaagcattggaaaatatagttttacttgatgaaaacaagaagaataagagtgatttacacgctacagcattgtcttttagacttttgaggcagcatggatttgaagtctcacaagacgtttttgaaagattcaaggacaaagaaggagggttttccggagaattaaaaggtgatgtccaaggtttattgagtttgtacgaagcttcttatttgggttttgaaggtgaaaacttattagaagaagctagaacattctccatcactcacttaaagaataatcttaaagagggaattaataccaaagttgccgaacaagtctcacatgcactagaattaccatatcatcaaagattgcaccgtttagaagctagatggttcttagacaagtatgaaccaaaggaacctcatcaccaattgttgttggaattagcaaagctagatttcaacatggtccaaacattgcaccaaaaggaacttcaggacctttctaggtggtggacagaaatgggattagccagtaagttggactttgtcagagataggttaatggaagtctatttttgggcactaggaatggcacctgatccacagtttggggagtgcagaaaagctgtaacaaaaatgttcggcttagtaacaattatagatgatgtgtacgacgtgtatggcacacttgacgaacttcaactttttactgatgccgttgagaggtgggatgtgaatgctattaatacattacccgattatatgaagttgtgcttcttagccttatataatactgtcaacgatacttcctatagtatcttaaaagagaagggtcacaataatcttagttatttgactaaaagttggcgtgaactttgtaaagcctttcttcaagaagctaagtggtctaataataagattattcctgccttttccaaatatcttgaaaacgcctctgtatcctcttccggtgttgcattgttagctccttcttatttctccgtttgccaacaacaggaagatatttcagatcacgccttgagatctcttacggattttcatggtttagtgcgtagtagttgcgttatttttcgtttgtgcaatgacttagctacttccgcagctgaattggaaagaggtgaaactaccaattcaattatctcttacatgcatgaaaatgatggcacatccgaagaacaggctcgtgaagagttaaggaagttgatcgacgcagagtggaagaaaatgaatagggagagagtcagtgattctacattgttaccaaaggcatttatggaaatagcagtaaacatggcaagagttagtcattgtacatatcaatacggtgatggcttggggcgtccagattacgctacagagaatagaattaagttattgttgattgatccttttccgattaaccaattaatgtacgtgtaaggccggccatccttgacaggcgcgcc

SEQ ID NO: 9 - DNA, 1751 bp, Artificial Sequence
  Description of Artificial SequenceSc-k-IspS alternate version 2 of Pueraria montana var. lobata kudzu vine isoprene synthase (IspS) codon-adjusted for expression in yeast
gcgatcgcacacacataaataaacaaaatgatatgtgcgacttcaagtcaattcactcagattaccgagcataactcgaggagatctgcgaattaccaacccaatttgtggaatttcgagtttcttcagtctctggaaaatgacttaaaggttgaaaaactagaagagaaagctactaagttagaagaagaagtcaggtgcatgataaacagggtggatactcaaccgttgagcttgttagaactaatagatgatgttcaaagattaggtctaacgtacaaattcgaaaaggacataatcaaagctctagaaaacatagtcttgcttgacgaaaacaaaaagaacaaaagtgacttacatgctaccgccttatcgtttagattgctgcgtcaacatggatttgaagtaagtcaagacgttttcgagagattcaaagataaagaaggcggtttttctggtgaattgaagggagatgtgcaagggctgttgtccttgtatgaggcttcctatctaggcttcgaaggcgaaaatctactggaagaggccagaacattcagcattacacatttgaagaataacttgaaagaggggattaacactaaggttgctgaacaggtatcccacgcactagaattaccatatcaccaaagattacaccgtttagaagcgagatggtttttggacaagtatgaaccaaaagaaccgcaccatcagcttcttttagagttggcaaaactagatttcaatatggttcagactttacatcaaaaagaattacaagatttgagcagatggtggaccgaaatgggacttgcctccaagttagactttgtgcgtgaccgtcttatggaagtttacttctgggccttagggatggcaccagatcctcagtttggtgaatgtaggaaagctgtcactaagatgtttggactggttacaatcattgatgatgtttacgatgtttatggaaccttagacgaattacaattgttcaccgatgcagtagagagatgggatgtgaatgccattaacacactgccagactacatgaagttgtgtttcttagctttgtacaacacagttaatgatacttcatattctattcttaaagagaaaggtcataacaacttatcctatttgacgaaatcatggagagagctatgtaaagctttcttacaagaagccaagtggtctaacaacaaaatcattcccgctttttcgaagtacttggagaatgcatctgttagttcatcaggtgtcgcgttactagcgcctagttacttttctgtctgccaacagcaagaagatatcagcgatcatgcattgaggtctttgaccgattttcatggcttagttagaagcagttgcgtcattttcaggctatgtaatgacttggctacatctgctgctgaacttgagcgtggtgagacaactaattccatcatttcgtatatgcacgaaaacgacggtacttctgaagaacaggccagagaagagttaagaaagttaatcgacgcagaatggaagaagatgaatagagagcgtgtgtcagattctacgttactgcctaaagcctttatggaaatagcagtgaatatggcaagagtatcacattgtacatatcaatacggtgacggtcttggtagacctgattatgcaacggaaaacagaatcaaattgcttcttattgatccatttcctatcaatcaactaatgtacgtataaggccggccatccttgacaggcgcgcc

SEQ ID NO: 10 - DNA, 1751 bp, Artificial Sequence
  Description of Artificial SequenceSc-k-IspS alternate version 3 of Pueraria montana var. lobata kudzu vine isoprene synthase (IspS) codon-adjusted for expression in yeast
gcgatcgcacacacataaataaacaaaatgatatgtgctacttcaagtcaattcactcagattaccgagcataactcgaggagatctgccaattaccaacccaatttgtggaatttcgagtttcttcagtctctggaaaatgacttaaaggttgaaaaactagaagagaaagctactaagttagaagaagaagtcaggtgcatgataaacagggtggatactcaaccgttgagcttgttagaactaatagatgatgttcaaagattaggtctaacgtacaaattcgaaaaggacataatcaaagctctagaaaacatagtcttgcttgacgaaaacaaaaagaacaaaagtgacttacatgctaccgccttatcgtttagattgctgcgtcaacatggatttgaagtaagtcaagacgttttcgagagattcaaagataaagaaggcggtttttctggtgaattgaagggagatgtgcaagggctgttgtccttgtatgaggcttcctatctaggcttcgaaggcgaaaatctactggaagaggccagaacattcagcattacacatttgaagaataacttgaaagaggggattaacactaaggttgctgaacaggtatcccacgcactagaattaccatatcaccaaagattacaccgtttagaagctagatggtttttggacaagtatgaaccaaaagaaccgcaccatcagcttcttttagagttggcaaaactagatttcaatatggttcagactttacatcaaaaagaattacaagatttgagcagatggtggaccgaaatgggacttgcctccaagttagactttgtgcgtgaccgtcttatggaagtttacttctgggccttagggatggcaccagatcctcagtttggtgaatgtaggaaagctgtcactaagatgtttggactggttacaatcattgatgatgtttacgatgtttatggaaccttagacgaattacaattgttcaccgatgcagtagagagatgggatgtgaatgccattaacacactgccagactacatgaagttgtgtttcttagctttgtacaacacagttaatgatacttcatattctattcttaaagagaaaggtcataacaacttatcctatttgacgaaatcatggagagagctatgtaaagctttcttacaagaagccaagtggtctaacaacaaaatcattcccgctttttcgaagtacttggagaatgcatctgttagttcatcaggtgtcgcgttactagcgcctagttacttttctgtctgccaacagcaagaagatatcagcgatcatgcattgaggtctttgaccgattttcatggcttagttagaagcagttgcgtcattttcaggctatgtaatgacttggctacatctgctgctgaacttgagcgtggtgagacaactaattccatcatttcgtatatgcacgaaaacgacggtacttctgaagaacaggccagagaagagttaagaaagttaatcgacgcagaatggaagaagatgaatagagagcgtgtgtcagattctacgttactgcctaaagcctttatggaaatagcagtgaatatggcaagagtatcacattgtacatatcaatacggtgacggtcttggtagacctgattatgcaacggaaaacagaatcaaattgcttcttattgatccatttcctatcaatcaactaatgtacgtataaggccggccatccttgacaggcgcgcc

SEQ ID NO: 11 - DNA, 1751 bp, Artificial Sequence
  Description of Artificial SequenceSc-k-IspS alternate version 4 of Pueraria montana var. lobata kudzu vine isoprene synthase (IspS) codon-adjusted for expression in yeast
gcgatcgcacacacataaataaacaaaatgatttgtgctacatcctcacaattcactcagataactgaacataactctagaagatcagccaattaccaacccaatttgtggaatttcgaattcttacagagtcttgagaatgatctaaaagtagaaaagttagaggaaaaagctacgaaacttgaagaagaggttagatgcatgattaacagagtagatacacagcctctatcattactagaacttatagatgacgtccaaagactaggtcttacttacaagtttgagaaagacataatcaaagccttagaaaacattgttcttttagatgaaaacaagaaaaacaagtctgatttgcacgctacggcattgagttttaggttgttgagacagcatggctttgaagtgtcacaagatgtttttgaaagattcaaagataaagaaggtggctttagtggggagttgaaaggtgacgtccagggcttactttctttgtacgaagcctcttatcttggtttcgaaggagaaaatctacttgaagaggccagaacattttcaattacccatttgaagaataacttgaaagagggtatcaatacaaaggttgcagagcaagtatctcatgctttagagctaccttatcatcaaagattgcatcgtttggaagctagatggtttctagataagtacgaaccaaaagaaccacaccatcaacttttgttagaattagccaagttggatttcaacatggtccagacacttcaccaaaaagagttacaagatttgtcaagatggtggaccgaaatgggtttagccagtaaactagacttcgtacgtgataggttaatggaggtatacttttgggccttagggatggctcctgacccacaatttggtgaatgtagaaaagctgttaccaaaatgtttggattagtaacaatcattgacgacgtttatgacgtttacggtactcttgatgaattacaattgtttaccgacgctgttgaacgttgggacgttaatgctatcaacactcttccagactatatgaaactttgctttttggcattgtataacacagttaatgatacgtcttactctattcttaaagaaaaaggtcacaataacttatcttacttgactaagtcttggagggaattgtgcaaagccttcttacaagaggcaaagtggtcaaacaataagatcattccggcattctccaagtatttggaaaacgctagtgtgtcctcatcaggagtggctttgttggcacctagttacttctctgtctgtcaacaacaagaggatatcagtgatcatgccctaaggtcattaacagattttcatggtttagtcagatcttcatgtgtcatattcagattgtgtaatgacttagcaacaagtgcagctgaattagaacgtggcgaaaccactaattccattatctcctatatgcacgagaatgatgggacttcagaagaacaggcaagggaggagttgcgtaaactaatcgatgctgaatggaagaaaatgaatagagaacgtgtgtctgattccaccctattaccaaaggctttcatggaaattgccgtgaatatggcaagggtttcccattgtacgtatcaatatggagatggattaggtaggcccgactatgctacagagaataggataaagctactattgatagatccatttccgattaaccagcttatgtatgtataaggccggccatccttgacaggcgcgcc

SEQ ID NO: 12 - protein, 595 aa, Populus tremuloides
  quaking aspen isoprene synthase (IspS)
MATELLCLHRPISLTHKLFRNPLPKVIQATPLTLKLRCSVSTENVSFSETETETRRSANYEPNSWDYDYLLSSDTDESIEVHKDKAKKLEAEVRREINNEKAEFLTLLELIDNVQRLGLGYRFESDIRRALDRFVSSGGFDGVTKTSLHGTALSFRLLRQHGFEVSQEAFSGFKDQNGNFLENLKEDIKAILSLYEASFLALEGENILDEAKVFAISHLKELSEEKIGKELAEQVSHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLIFAYFAVVQNIKKEEIENLQKYHDIISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 13 - protein, 595 aa, Populus nigra
  Lombardy poplar isoprene synthase (IspS)
MATELLCLHRPISLTHKLFRNPLPKVIQATPLTLKLRCSVSTENVSFTETETETRRSANYEPNSWDYDYLLSSDTDESIEVYKDKAKKLEAEVRREINNEKAEFLTLPELIDNVQRLGLGYRFESDIRRALDRFVSSGGFDAVTKTSLHATALSFRLLRQHGFEVSQEAFSGFKDQNGNFLKNLKEDIKAILSLYEASFLALEGENILDEAKVFAISHLKELSEEKIGKDLAEQVNHALELPLHRRTQRLEAVWSIEAYRKKEDADQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAIDDLPDYMKLCFLALYNTINEIAYDNLKDKGENILPYLTKAWADLCNAFLQEAKWLYNKSTPTFDEYFGNAWKSSSGPLQLVFAYFAVVQNIKKEEIDNLQKYHDIISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 14 - protein, 7 aa, Artificial Sequence
  Description of Artificial Sequencepoplar and aspen isoprene synthase (IspS) mature protein N-terminal residues
CSVSTEN

SEQ ID NO: 15 - protein, 6 aa, Artificial Sequence
  Description of Artificial Sequencekudzu isoprene synthase (IspS) mature protein N-terminal residues
CATSSQ

SEQ ID NO: 16 - DNA, 34 bp, Artificial Sequence
  Description of Artificial Sequencesynthetic glyceraldehyde-3-phosphate dehydrogenase (GDP) promoter-F PCR forward primer
tgcgcggccgctcgagtttatcattatcaatact

SEQ ID NO: 17 - DNA, 34 bp, Artificial Sequence
  Description of Artificial Sequencesynthetic glyceraldehyde-3-phosphate dehydrogenase (GDP) promoter-R PCR reverse primer
ctagcgatcgcttattcgaaactaagttcttggt

SEQ ID NO: 18 - DNA, 32 bp, Artificial Sequence
  Description of Artificial Sequencesynthetic N-5′-phosphoribosyl-anthranilate isomerase (TRP1d)-F PCR forward primer
ggattaattaaagcacgtgagtatacctgatt

SEQ ID NO: 19 - DNA, 47 bp, Artificial Sequence
  Description of Artificial Sequencesynthetic N-5′-phosphoribosyl-anthranilate isomerase (TRP1d)-R PCR reverse primer
ttagcggccgctttcaaaacgcctgctggcaagtgcacaaacaatac

SEQ ID NO: 20 - DNA, 39 bp, Artificial Sequence
  Description of Artificial Sequencesynthetic iso-1-cytochrome c (CYC1) terminator-F PCR forward primer
cccggccggcctttgtcgatatcatgtaattagttatgt

SEQ ID NO: 21 - DNA, 36 bp, Artificial Sequence
  Description of Artificial Sequencesynthetic iso-1-cytochrome c (CYC1) terminator-R PCR reverse primer
cgggcgatcgcaagcatgcaaattaaagccttcgag

SEQ ID NO: 22 - DNA, 27 bp, Artificial Sequence
  Description of Artificial Sequencesynthetic ribosomal DNA (rDNA) rDNA1-F PCR forward primer
gtacctgcaggatgagagtagcaaacg

SEQ ID NO: 23 - DNA, 27 bp, Artificial Sequence
  Description of Artificial Sequencesynthetic ribosomal DNA (rDNA) rDNA1-R PCR reverse primer
tgattaattaaccgggtaacccagttc

SEQ ID NO: 24 - DNA, 27 bp, Artificial Sequence
  Description of Artificial Sequencesynthetic ribosomal DNA (rDNA) rDNA2-F PCR forward primer
gttgcgatcgccacctgtcactttgga

SEQ ID NO: 25 - DNA, 27 bp, Artificial Sequence
  Description of Artificial Sequencesynthetic ribosomal DNA (rDNA) rDNA2-R PCR reverse primer
ttaggcgcgcccaggttccaccaaaca

SEQ ID NO: 26 - DNA, 29 bp, Artificial Sequence
  Description of Artificial Sequencesynthetic beta-isopropylmalate dehydrogenase (LEU2d)-F PCR forward primer
ggattaattaaatatatatttcaaggata

SEQ ID NO: 27 - DNA, 41 bp, Artificial Sequence
  Description of Artificial Sequencesynthetic beta-isopropylmalate dehydrogenase (LEU2d)-R PCR reverse primer
gaagcggccgcataaagtttctgtacaaatatcataaaaaa

SEQ ID NO: 28 - DNA, 41 bp, Artificial Sequence
  Description of Artificial Sequencesynthetic kIspS-F PCR forward primer
cagcgatcgcacacacataaataaacaaaatgccatggatt

SEQ ID NO: 29 - DNA, 45 bp, Artificial Sequence
  Description of Artificial Sequencesynthetic kIspS-R PCR reverse primer
taggcgcgcctgtcatggatggccggccttacacgtacattagtt

SEQ ID NO: 30 - DNA, 19 bp, Artificial Sequence
  Description of Artificial Sequenceportion of GDP promoter region missing from synthetic ATCC plasmid for Sc-k-IspS alternate versions of Pueraria montana var. lobata kudzu vine isoprene synthase (IspS) codon-adjusted for expression in yeast
acacacataaataaacaaa

SEQ ID NO: 31 - DNA, 10 bp, Artificial Sequence
  Description of Artificial Sequencesynthetic spacer between FseI and AscI restriction sites in Sc-k-IspS alternate versions of Pueraria montana var. lobata kudzu vine isoprene synthase (IspS) codon-adjusted for expression in yeast
atccttgaca

(19) United States
(12) Patent Application Publication
(10) Pub. No.: US 2011/0045563 A1
(43) Pub. Date: Feb. 24, 2011

(54) SHORT CHAIN VOLATILE ISOPRENE HYDROCARBON PRODUCTION USING THE MEVALONIC ACID PATHWAY IN GENETICALLY ENGINEERED YEAST AND FUNGI

(75) Inventor: Anastasios Melis, El Cerrito, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/866,016

(22) PCT Filed: Feb. 5, 2009
(86) PCT No.: PCT/US09/33235

§ 371 Date: Oct. 18, 2010

Related U.S. Application Data
(60) Provisional application No. 61/026,647, filed on Feb. 6, 2008.

(51) Int. Cl. C12P 5/02; C12N 1/19; C12N 1/15

(52) U.S. Cl. 435/167; 435/254.2; 435/254.11

(57) ABSTRACT

The present invention provides methods and compositions for producing isoprene hydrocarbons from Ascomycota such as yeast and filamentous fungi.

### CROSS-REFERENCE TO RELATED APPLICATIONS

This application claims benefit of U.S. provisional application No. 61/026,647, filed Feb. 6, 2008, which application is herein incorporated by reference.

### BACKGROUND OF THE INVENTION

A variety of herbaceous, deciduous and conifer plants are known to possess the genetic and enzymatic capability for the synthesis and release of short-chain isoprenoids (e.g., isoprene (C₅H₈) and methyl-butenol (C₅H₁₀O)) into the surrounding environment. Such short-chain isoprenoids are derived from small organic molecules that are products of cellular or chloroplast metabolism, and can be synthesized in the chloroplast of herbaceous, deciduous and conifer plants at substantial rates under certain environmental stress conditions. Heat-stress of the organism is particularly important for the induction of this process in herbaceous, deciduous and conifer plants.

Emission of isoprene from herbaceous, deciduous, and conifer plants is due to the presence of an isoprene synthase (IspS) gene, a nuclear gene encoding for a chloroplast-localized protein that catalyzes the conversion of isopentenyl diphosphate (IPP) or dimethylallyl diphosphate (DMAPP) to isoprene. Five-carbon isoprenoids, e.g. isoprene (C₅H₈) and methyl-butenol (C₅H₁₀O), are relatively small hydrophobic molecules, synthesized directly from IPP or DMAPP (FIG. 1). These isoprenoids are volatile molecules that easily go through cellular membranes and thereby are emitted from the leaves into the atmosphere. The process of heat stress-induction and emission of short chain hydrocarbons by plants has been discussed as undesirable pollution of the atmosphere in the literature. There has been no description of the mass-generation, harvesting and sequestration of these hydrocarbons from the leaves of herbaceous, deciduous and conifer plants.

The universal precursors of all isoprenoids in all living organisms are two isomeric 5-carbon molecules, IPP and DMAPP. IPP and DMAPP can be synthesized through two distinctly different biosynthetic pathways, operating in separate cells types or cellular compartments:
(i) The Mevalonic Acid (MVA) pathway functions mainly in the cytosol of Eukaryotes and Archaea, including Fungi/Ascomycota, e.g., yeast and filamentous fungi (FIG. 2). MVA precursor molecules are Acetyl-CoA forming from co-enzyme A in the cells and acetic acid, which is the product of sugar and other substrate catabolism. Mevalonic acid is the first dedicated precursor of IPP and DMAPP in the cytosol of all Eukaryotes and Archaea, leading to the synthesis of all isoprenoids in Fungi/Ascomycota, e.g., Saccharomyces cerevisiae and Neurospora crassa (FIG. 2). MVA precursor molecules are Acetyl-CoA forming (via the enzyme Thiolase) Acetoacetyl-CoA, forming (via the HMG-CoA synthase) 3-hydroxy-3-methyl-glutaryl-CoA (HMG-CoA), forming (via the HMG-CoA reductase) Mevalonic Acid. Mevalonic acid is the first dedicated precursor of IPP and DMAPP in the cytosol of all Eukaryotes and Archaea (FIG. 3).
(ii) The DXP-MEP pathway of isoprenoid biosynthesis functions in the plastids, i.e., chloroplasts, of plant cells and eukaryotic microalgae and in the cytosol of Eubacteria (e.g., Escherichia coli, Rhodospirillum rubrum, Synechocystis sp.).

Different organisms may possess both isoprenoid biosynthetic pathways, or employ only one of the two pathways for the biosynthesis of their isoprenoids. For example, higher plant cells possess and employ both the cytosolic MVA and plastidic DXP-MEP pathways for the synthesis of different isoprenoids. Unicellular green algae employ the plastidic DXP-MEP pathway only. Conversely, yeast and fungi such as Saccharomyces cerevisiae and Neurospora crassa, respectively, model organisms useful in fermentations, possess the MVA biosynthetic pathway only for the biosynthesis of isoprenoids.

There is an urgent need for the development of renewable biofuels that will help meet global demands for energy but without contributing to climate change. The current invention addresses this need by providing methods and compositions to renewably generate volatile short-chain hydrocarbons that are derived upon the fermentation of sugar, starch, and other cellular metabolites by yeast or fungi. Such hydrocarbons can serve as biofuels or feedstock in the synthetic chemistry industry. The present invention is based on the discovery that the MVA pathway in yeast and fungi can be manipulated to generate volatile isoprene hydrocarbons.

### BRIEF SUMMARY OF THE INVENTION

The present invention provides methods and compositions for the genetic modification of the mevalonic acid pathway in Fungi/Ascomycota type of microorganisms (e.g., yeast or filamentous fungi) for the production of volatile isoprene hydrocarbons. The wild-type strains of these microorganisms are incapable of performing this hydrocarbon production, as they lack the gene/enzyme that catalyzes the respective reaction. The invention confers to these microorganisms heterologous expression of an isoprene synthase gene, e.g., from kudzu (Pueraria montana, GenBank Accession # AY316691) or poplar (Populus species, GenBank Accession # AY341431, AM084344, AB198180, AJ294819). Such genetic modification enables these microorganisms (yeast, fungi) to produce isoprene, a 5-carbon volatile hydrocarbon, as a side product of the isoprenoid biosynthetic pathway. Harvesting of isoprene is implemented upon organism cultivation in a bioreactor.

The invention is based, in part, on the discovery that Fungi/Ascomycota cells such as yeast and filamentous fungi can be genetically modified to produce 5-carbon isoprenoids (e.g., FIGS. 1 and 3). The MVA isoprenoid biosynthetic pathway is absolutely required in yeast and fungi, as it leads to the synthesis of many longer-chain isoprenoids that serve as essential cellular compounds. Yeast and fungi cells specifically encode for this pathway in the nucleus and express it in the cytosol (FIG. 2), where they utilize the corresponding enzymes (FIG. 3) for the biosynthesis of a variety of isoprenoid molecules. The present invention relates to methods and compositions for the use of genetically modified yeast and fungi in the production and harvesting of 5-carbon volatile isoprenoid compounds, e.g., isoprene and methyl-butenol (FIG. 1). Such genetically modified organisms can be used commercially in a contained mass culture system, e.g., a fermentor, to provide a source of renewable fuel for internal combustion engines or, upon on-board reformation, in fuel-cell operated engines; or to provide a source of isoprene hydrocarbons for use in other chemical processes such as chemical synthesis.

Yeast and fungi do not possess an isoprene synthase or a methyl-butenol synthase gene, which catalyze the last committed step in isoprene and methyl-butenol biosynthesis, respectively. This invention therefore provides methods and compositions to genetically modify yeast and fungi to express an isoprene synthase gene, e.g., a codon-adjusted poplar or kutzu isoprene synthase gene, to produce isoprene.

In additional aspects, the invention also provides method and compositions for the genetic modification of yeast and fungi to over-express endogenous genes and proteins encoding the first committed step in MVA pathway isoprenoid biosynthesis. The invention can thus further comprise increasing expression of native HMG-CoA synthase and/or HMG-CoA reductase genes in yeast or fungi, e.g., Saccharomyces cerevisiae, Neurospora crassa, or Aspergillus fumigatus. HMG-CoA synthase and HMG-CoA reductase are enzymes that catalyze the first committed steps in isoprenoid biosynthesis (FIG. 3).

In some embodiments, yeast or fungi, e.g., Saccharomyces cerevisiae, a species of budding yeast; or Neurospora crassa or Aspergillus fumigatus, filamentous fungi, are employed.

Yeast/fungal metabolism can be directed toward volatile isoprene hydrocarbon generation; they grow and reproduce faster than terrestrial or aquatic plants, doubling of biomass per day; their biomass is non-toxic and non-polluting, thus environmentally friendly for mass cultivation and commercial exploitation. Accordingly, in some embodiments, the invention provides a process to modify the highly efficient metabolism of Fungi/Ascomycota to generate, in high volume, short-chain isoprene hydrocarbons (e.g., C₅H₈) from organic matter feedstock, i.e., small organic molecules (acetate), sugars, starch, or other cellular compounds and metabolites. Genetically modified yeast or fungi can metabolize such feedstock (usually sucrose) or starch (polymerized glucose) or biomass derived from agricultural or wild land plants, such as found in crushed grain, corn, potatoes or cellulose, and to release volatile isoprene hydrocarbons. Such modified yeast or fungi can be grown in large capacity (e.g. 1,000-1,000,000 liters), in fully enclosed bioreactors/fermentors for the production and harvesting of volatile short-chain isoprene hydrocarbons.

The invention will help eliminate a number of current barriers in the commercial production, storage and utilization of renewable energy, including: (a) lowering the cost of production and storage of fuel; (b) improving fuel Weight/Volume ratios; (c) improving the efficiency of fuel production/storage; (d) increasing the durability of fuel storage; (e) minimizing auto-refueling time; (f) offering sufficient fuel storage for acceptable vehicle range; (g) producing a fuel amenable to regeneration process; (h) producing a fuel that is not subject to interference by air or carbon dioxide in either the production or storage stage.

In one aspect, the invention provides a method of producing isoprene hydrocarbons in a Fungi/Ascomycota microorganism, the method comprising: introducing an expression cassette that comprises a nucleic acid sequence encoding isoprene synthase into the microorganism; and culturing the microorganism under conditions in which the nucleic acid encoding isoprene synthase is expressed. In some embodiments, the microorganism is a yeast such as Saccharomyces cerevisiae. In alternative embodiments, the microorganism is a a filamentous fungus, e.g., Neurospora crassa or Aspergillus fumigatus.

In some embodiments, the DNA nucleic acid introduced into the yeast or fungus comprises a codon-adjusted sequence such as the sequence set forth in SEQ ID NO:3 or SEQ ID NOs. 8-11. SEQ ID NO:3, and SEQ ID NOs. 8-11 each provide the coding sequence of an isoprene synthase gene, codon adjusted for expression in yeast. The nucleic acid sequences encode an isoprene synthase polypeptide that has the sequence set forth in SEQ ID No:5. The sequence set forth in SEQ ID NO:5 lacks a transit peptide region in its N-terminus, thus, upon translation termination, the mature polypeptide will remain and function in the cytosol of the yeast cell.

In some other embodiments, the DNA nucleic acid introduced into the yeast or fungus comprises a codon-adjusted sequence that has the sequence set forth in SEQ ID NO:4. SEQ ID NO:4 provides the coding sequence of an isoprene synthase gene that is codon-adjusted for expression in Neurospora crassa. This exemplary isoprene synthase cDNA encodes a polypeptide that has the sequence set forth in SEQ ID NO:5. The sequence set forth in SEQ ID NO:5 lacks a transit peptide region in its N-terminus, thus, upon translation termination, the mature polypeptide will remain and function in the cytosol of the yeast cell.

In another aspect, the invention provides a yeast or fungus microorganism that expresses isoprene synthase, wherein the microorganism comprises a heterologous nucleic acid that encodes isoprene synthase and that is operably linked to a promoter. The promoter can be a constitutive promoter or an inducible promoter. In some embodiments, the microorganism is Saccharomyces cerevisiae, Neurospora crassa, or Aspergillus fumigatus. In some embodiments, the heterologous nucleic acid comprises a sequence that encodes an isoprene synthase comprising the sequence set forth in SEQ ID NO:5.

In a further aspect, the invention provides a method of producing isoprene hydrocarbons in a yeast or a fungus, e.g., a filamentous fungus, that comprises a heterologous gene that encodes isoprene synthase, the method comprising mass-culturing the yeast or fungus in an enclosed bioreactor/fermentor under conditions in which the isoprene synthase gene is expressed, and harvesting isoprene hydrocarbons produced by the yeast or fungus. In some embodiments, the Fungi/Ascomycota is Saccharomyces cerevisiae, Neurospora crassa, or Aspergillus fumigatus.

### BRIEF DESCRIPTION OF THE DRAWINGS

FIG. 1. Schematic that depicts the single step enzymatic reaction for the biosynthesis of isoprene and methyl-butenol in the chloroplast of herbaceous/deciduous trees and pines, respectively. The enzymes involved (isoprene synthase, MBO synthase) and the chemical formulae of isoprene (C₅H₈) and methyl-butenol (C₅H₁₀O) are also shown.

FIG. 2. Schematic that depicts the eukaryotic nuclear-encoded and cytosolically (Ct)-localized mevalonic acid pathway for the synthesis of IPP, DMAPP and a variety of isoprenoids, occurring in yeast, fungi, plant cells, animals and Archaea.

FIG. 3. Schematic that depicts the specific reaction steps and enzymes involved in the mevalonic acid pathway in Fungi/Ascomycota, leading to IPP and DMAPP biosynthesis.

FIG. 4. Alignment of exemplary isoprene synthase proteins.

FIG. 5. Restriction endonuclease map of plasmid construct pSckIspS. The 1.7 kb S. cerevisiae codon-optimized kudzu SckIspS was synthesized and assembled in a kanamycin resistance containing plasmid pSckIspS.

FIG. 6. Restriction endonuclease map of plasmid construct pSckIspStg. The 0.65 kb GPD promoter (glyceraldehyde-3-phosphate dehydrogenase) was fused to the 1.7 kb S. cerevisiae codon-optimized kudzu SckIspS, followed by the 0.26 kb CYC1 (iso-1-cytochrome c) terminator. The 0.8 kb TRP1d (N-5′-phosphoribosyl-anthranilate isomerase) fragment was inserted as a selectable marker for transformation of S. cerevisiae. The 0.59 kb and 0.67 kb rDNA (ribosomal DNA) fragments were included to faciliate plasmid integration into the yeast genomic DNA by the process of homologous recombination. TRP1d, the N-(5′-phosphoribosyl)anthranilate isomerase, is one of the required enzymes in the tryptophan biosynthetic pathway; SckIspS: S. cerevisiae codon-optimized kudzu vine isoprene synthase.

FIG. 7. Restriction endonuclease map of plasmid pSckIspSlg. The 1.2 kb LEU2d (β-isopropylmalate dehydrogenase) fragment replaced the 0.8 kb TRP1d in the pSckIspStg plasmid (compare with FIG. 5) and was used as a selectable marker for transformation of S. cerevisiae. Abbreviations are as follows: rDNA, ribosomal DNA; LEU2d, β-isopropylmalate dehydrogenase (one of the required enzymes in the leucine biosynthetic pathway); GPD, glyceraldehyde-3-phosphate dehydrogenase; SckIspS: S. cerevisiae codon-optimized kudzu vine isoprene synthase; CYC1, iso-1-cytochrome c.

FIG. 8. Map of the 9.1 kb rDNA repeat unit in Saccharomyces cerevisiae (baker's yeast). The yeast rDNA consists of 100-200 tandem repeat copies of a 9.1 kb unit on the right arm of chromosome XII. 5S, 18S, 5.8S, and 25S rRNAs are transcribed from the rDNA by RNA polymerase. Each rRNA is present as a single copy in a 80S yeast ribosome. Abbreviations used are as follows: NTS, non-transcribed spacer; ITS, internal transcribed spacer; ETS, external transcribed spacer;
rRNA: 5S, 5.8S, 25S, and 18S rRNA
Spacer region: ITS1, ITS2, 5′ETS, 3′ETS, NTS1, and NTS2
18S, 5.8S, and 25S rRNA are transcribed from 35S pre-rRNA
5S rRNA transcribed separately
40S ribosomal subunit: 18S rRNA
60S ribosomal subunit: 5S, 5.8S, and 25S rRNA
80S yeast ribosome (60S+40S): each copy of 5S, 5.8S, 18S, and 25S rRNA+78 r-proteins

FIG. 9. rDNA location on Saccharomyces cerevisiae chromosome XII. The top panel shows a map of chromosome XII. The solid circle near the SSA2 shows the position of the chromosome's centromere. The relevant open reading frame, marked on chromosome XII by a rectagle, is shown in greater detail in the below diagrams under the solid line. For simplification, only two sets of rDNA copies are shown. Abbreviations are as follows: RDN37, 37S rDNA; RDN5, 5S rDNA.

FIG. 10. Example of gas chromatographic analysis of the headspace of wild type and IspS transformant cultures. Cells were grown on YPD liquid media till the end of the exponential growth phase. Cultures were sealed with silicon stoppers and incubated under the same growth conditions for 24 h. Subsequently, one (1) ml gaseous samples were withdrawn from the headspace of the flasks and analyzed by GC.

### DETAILED DESCRIPTION OF THE INVENTION

### Introduction

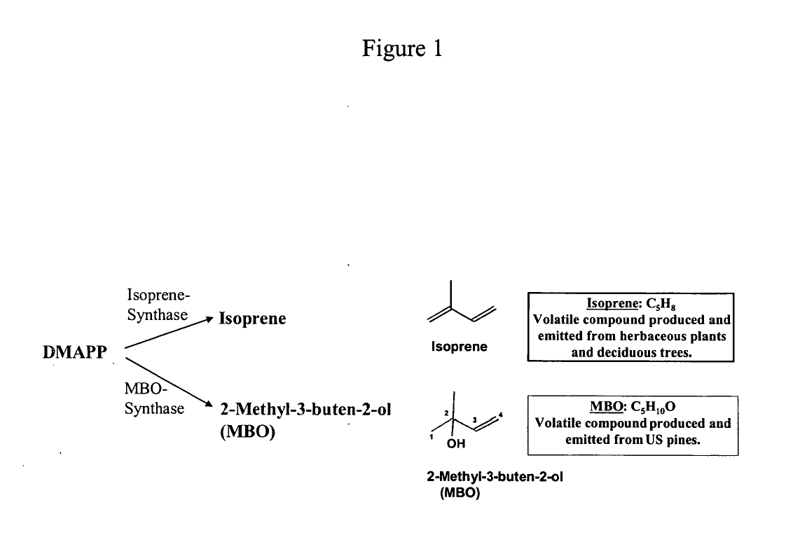

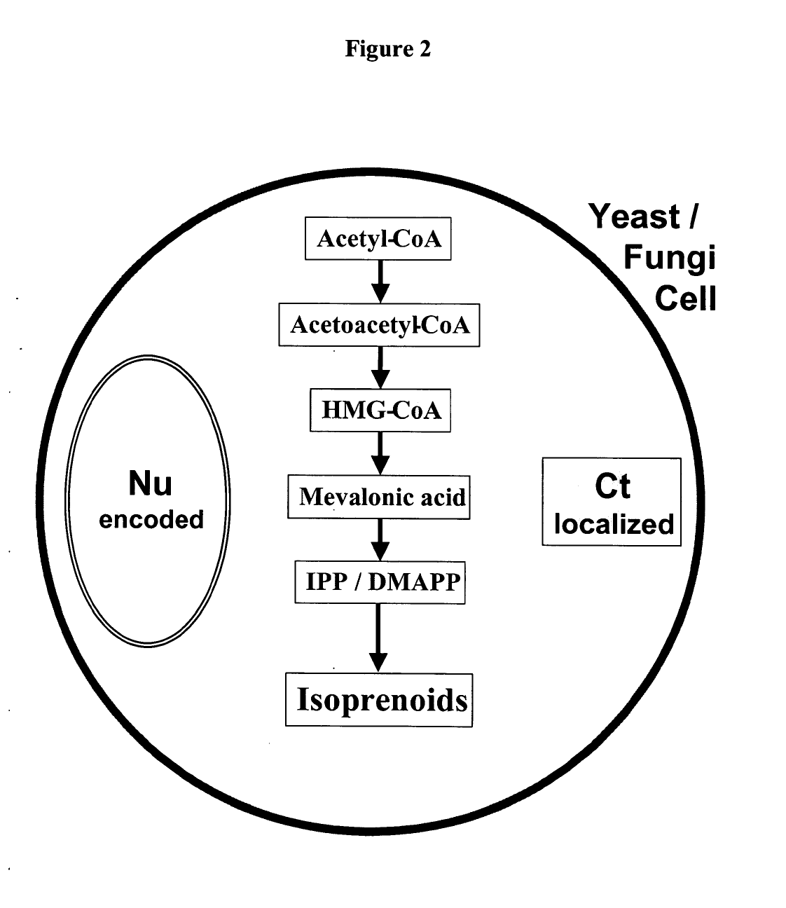

Small 5-carbon isoprenoids, e.g., isoprene (C₅H₈) and methyl-butenol (C₅H₁₀O), are relatively small hydrophobic molecules, synthesized directly from IPP or DMAPP (FIG. 1). These isoprenoids are volatile molecules that easily go through cellular membranes and thereby are emitted into the atmosphere. The relative volatility of these compounds is an asset, as it permits easy separation of these products from the cells and from the surrounding growth medium. Moreover, condensation of isoprenoids into liquid form can be readily achieved upon low-level compression.

This invention provides for the use of genetically modified microorganisms in the commercial production and harvesting of such 5-carbon volatile isoprenoid compounds, e.g., isoprene (C₅H₈), via the MVA biosynthetic pathway. It employs molecular genetic and transformation technologies to endow Ascomycota/Fungi, e.g. yeast or filamentous fungi, with the ability to express isoprene synthase or methyl-butenol synthase, thereby converting them into commercially viable isoprenoid producers.

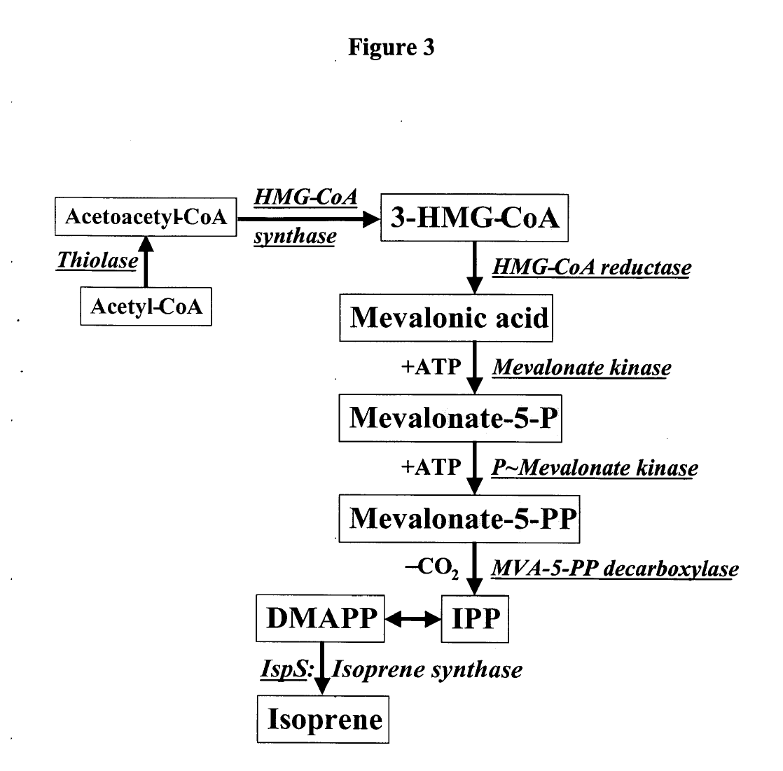

The MVA isoprenoid biosynthetic pathway is absolutely required in Ascomycota/Fungi such as yeast or filamentous fungi, as they lack the DXP-MEP pathway and must rely on the MVA pathway for the synthesis of all essential cellular isoprenoids. Accordingly, the MVA isoprenoid pathway in these organisms is specifically responsible for the biosynthesis of a great variety of isoprenoid molecules (carotenoids, tocopherols, phytol, sterols, hormones, among many others). The current invention manipulates the MVA pathway in these organisms to mass-produce 5-carbon isoprenoids (e.g. FIGS. 1 and 3).

Unlike many herbaceous, deciduous and conifer plants, Ascomycota/Fungi, e.g. yeast such as Saccharomyces cerevisiae or filamentous fungi such as Neurospora crassa or Aspergillus fumigatus, are not endowed with the last step in volatile isoprenoid biosynthesis, i.e., they do not possess the isoprene synthase or methyl-butenol synthase genes, which catalyze the last committed step in isoprene (C₅H₈) and methyl-butenol (C₅H₁₀O) biosynthesis, respectively. This invention provides method and compositions for the genetic transformation of yeast and fungi, e.g., Saccharomyces cerevisiae, Neurospora crassa, or Aspergillus fumigatus with an isoprene synthase gene, e.g., a codon-adjusted poplar or kudzu isoprene synthase gene, so as to confer isoprene (C₅H₈) production to these eukaryotic microorganisms. This invention also contemplates genetic modification of Ascomycota/Fungi, e.g., yeast or filamentous fungi, with a codon-adjusted pine methyl-butenol synthase gene, so as to confer the last committed step in methyl-butenol (C₅H₁₀O) production to these eukaryotic microorganisms.

### Definitions

In the context of this invention “Ascomycota/Fungi” or “Ascomycota” refer to members of the Ascomycota, which is a phylum of the kingdom Fungi. Ascomycota are commonly known as the Sac Fungi. Representative groups of Ascomycota include, e.g., Neurospora, Penicillium, Aspergillus and the yeasts (e.g., Candida, Kluyveromyces, Saccharomyces, Schizosaccharomyces, Pichia and Yarrowia). The Acomycota used in the invention are typically microorganisms, e.g., yeasts and filamentous fungi such as Neurospora and Aspergillus.

A “filamentous fungus” in the context of this invention refers to a eukaryotic microorganism and includes all filamentous forms of the Ascomycota. These fungi are characterized by multicellular hyphae forming a mycelium. Vegetative growth of filamentous fungi is by hyphal elongation.

A “volatile isoprene hydrocarbon” in the context of this invention refers to a 5-carbon, short chain isoprenoid, e.g., isoprene or methyl-butenol.

The terms “nucleic acid” and “polynucleotide” are used synonymously and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones. Thus, nucleic acids or polynucleotides may also include modified nucleotides, that permit correct read through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell. In the context of this invention, the term “IspS coding region” when used with reference to a nucleic acid reference sequence such as SEQ ID NO:1, SEQ ID NO:3, SEQ ID NO:4, SEQ ID NO:6, or SEQ ID NOs. 8-11 refers to the region of the nucleic acid that encodes the protein.

An IspS “gene” in the context of this invention refers to a nucleic acid that encodes an isoprene synthase (IspS) protein, or fragment thereof. Thus, such a gene is often a cDNA sequence that encodes IspS. In other embodiments, an IspS gene may include sequences such as introns that are not present in a cDNA.

The term “promoter” or “regulatory element” refers to a region or sequence determinants located upstream or downstream from the start of transcription that direct transcription. As used herein, a promoter includes necessary nucleic acid sequences near the start site of transcription, such as, in the case of a polymerase II type promoter, a TATA element. A promoter also optionally includes distal elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter) and a second nucleic acid sequence, such as an IspS gene, wherein the expression control sequence directs transcription of the nucleic acid corresponding to the second sequence. A “yeast promoter” or “fungus promoter” is a promoter capable of initiating transcription in yeast and/or fungus cells, respectively. Such a promoter is therefore active in a yeast cell or fungus cells, but need not originate from that organism. It is understood that limited modifications can be made without destroying the biological function of a regulatory element and that such limited modifications can result in regulatory elements that have substantially equivalent or enhanced function as compared to a wild type regulatory element. These modifications can be deliberate, as through site-directed mutagenesis, or can be accidental such as through mutation in hosts harboring the regulatory element. All such modified nucleotide sequences are included in the definition of a yeast or filamentous fungus regulatory element as long as the ability to confer expression in the yeast or fungus is retained.

The term “mevalonate pathway” or “mevalonic acid pathway” or “MVA pathway” is used herein to refer to the biosynthetic pathway that converts acetyl-CoA to IPP through a MVA pathway intermediate (FIG. 3).

“Increased” or “enhanced” activity or expression of a MVA pathway gene refers to an increase in activity of an enzyme in the pathway, e.g., HMG-CoA synthase or HMG-CoA reductase. Examples of such increased activity or expression include the following. Enzyme activity or expression of a gene encoding the enzyme is increased above the level of that in wild-type, non-transgenic control microorganism (i.e., the quantity of enzyme activity or expression of the gene encoding the enzyme is increased). Enzyme activity or expression of a gene encoding the enzyme is also considered to be increased in expression in a cell when it is not normally detected in wild-type, non-transgenic cells. Enzyme activity or expression is also considered to be increased when enzyme activity or expression of a gene encoding the enzyme is present in a cell for a longer period than in a wild-type, non-transgenic controls (i.e., duration of enzyme activity or expression of a gene encoding the enzyme is increased).

“Expression” of an IspS gene in the context of this invention typically refers to introducing an IspS gene into a Ascomycota microorganism e.g., yeast or a fungus, e.g., a filamentous fungus, in which it is not normally expressed. Accordingly, an “increase” in IspS activity or expression is generally determined relative to wild type cells, e.g., yeast or a filamentous fungus, that have no IspS activity.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants

An “IspS polynucleotide” is a nucleic acid sequence that comprises the IspS coding region of SEQ ID NO:1, SEQ ID NO:3, SEQ ID NO:4, or SEQ ID NO:6, or any one of SEQ ID NOs 8-11; or a nucleic acid sequence that is substantially similar to the IspS-coding region of SEQ ID NO:1, SEQ ID NO:3, SEQ ID NO:4, or SEQ ID NO:6, or any one of SEQ ID NOs 8-11; or a nucleic acid sequence that encodes a polypeptide of SEQ ID NO:5 or SEQ ID NO:7, or a polypeptide that is substantially similar to SEQ ID NO:5 or SEQ ID NO:7, or a domain thereof that has IspS activity. Thus, an IspS polynucleotide: 1) comprises a region of about 100, 150, 200, 300, 500, 1,000, or 1500, or more nucleotides of the IspS coding region of SEQ ID NO:1, SEQ ID NO:3, SEQ ID NO:4, or SEQ ID NO:6, or any one of SEQ ID NOs 8-11; or 2) hybridizes to the IspS coding region of SEQ ID NO:1, SEQ ID NO:3, SEQ ID NO:4, or SEQ ID NO:6, or any one of SEQ ID NOs 8-11, or the complements thereof, under stringent conditions, or 3) encodes an IspS polypeptide or fragment having IspS activity of at least 50 contiguous amino acids, typically of at least 100, 150, 200, 250, 300, 350, 400, 450, 500, or 550, or more contiguous residues of an IspS polypeptide, e.g., SEQ ID NO:2, SEQ ID NO:5 or SEQ ID NO:7; or 4) encodes an IspS polypeptide or fragment that has at least 55%, often at least 60%, 65%, 70%, 75%, 80%, 85%, 90%, 95%, or greater identity to SEQ ID NO:2, SEQ ID NO:5, or SEQ ID NO:7; or to a comparison window of at least 100, 200, 300, 400, 500, or 550 contiguous amino acid residues of SEQ ID NO:2, SEQ ID NO:5 or SEQ ID NO:7; or 5) has a nucleic acid sequence that has greater than about 60%, 65% , 70%, 75%, 80%, 85%, 90%, 95%, 96%, 97%, 98%, 99%, or higher nucleotide sequence identity to SEQ ID NO:1, SEQ ID NO:3, SEQ ID NO:4, or SEQ ID NO:6, or any one of SEQ ID NOs 8-11; or at least 80%, 85%, 90%, or at least 95%, 96%, 97%, 98%, 99%, or greater identity over a comparison window of at least 100, 200, 500, 1000, or more nucleotides of the IspS coding region of SEQ ID NO:1, SEQ ID NO:3, SEQ ID NO:4, or SEQ ID NO:6, or any one of SEQ ID NOs 8-11; or 6) is amplified by primers that amplify the IspS coding region of SEQ ID NO:1, SEQ ID NO:3, SEQ ID NO:4, or SEQ ID NO:6, or any one of SEQ ID NOs 8-11. The term “ IspS polynucleotide” refers to double stranded or singled stranded nucleic acids. The IspS nucleic acids for use in the invention encode an active IspS that catalyzes the conversion of IPP or DMAPP substrate to isoprene.

An “ IspS polypeptide” is an amino acid sequence that comprises the amino acid sequence of SEQ ID NO:2, SEQ ID NO:5 or SEQ ID NO:7, or is substantially similar to SEQ ID NO:2, SEQ ID NO:5 or SEQ ID NO:7, or a fragment or domain thereof that has isoprene synthase activity. Thus, an IspS polypeptide can: 1) have at least 55% identity, typically at least 60%, 65%, 70%, 75%, 80%, 85%, 90%, 95% or greater identity to SEQ ID NO:2, SEQ ID NO:5 or SEQ ID NO:7, or over a comparison window of at least 100, 200, 250, 300, 250, 400, 450, 500, or 550 amino acids of SEQ ID NO:2, SEQ ID NO:5 or SEQ ID NO:7; or 2) comprise at least 100, typically at least 200, 250, 300, 350, 400, 450, 500, 550, or more contiguous amino acids of SEQ ID NO:2, SEQ ID NO:5 or SEQ ID NO:7; or 3) bind to antibodies raised against an immunogen comprising an amino acid sequence of SEQ ID NO:2, SEQ ID NO:5 or SEQ ID NO:7. An IspS polypeptide in the context of this invention is a functional protein that catalyzes the conversion of IPP or DMAPP to isoprene.

As used herein, a homolog or ortholog of a particular IspS gene (e.g., SEQ ID NO:1 or SEQ ID NO:6) is a second gene in the same plant type or in a different plant type that is substantially identical (determined as described below) to a sequence in the first gene.

“HMG-CoA synthase” and “HMG-CoA reductase” nucleic acids and polypeptide refer to fragments, variants, and the like. Exemplary HMG CoA synthase protein sequences include NP_{—}013580, Saccharomyces cerevisiae; XP_{—}754553, Aspergilllus fumigatus; and EAA28325, Neurospora crassa. Exemplary HMG CoA reductase polypepitdes include NP_{—}013636, Saccharomyces cerevisiae (Hmg1P); NP_{—}013555, Saccharomyces cerevisiae (Hmg2P); XP_{—}749502, Aspergilllus fumigatus; and EAA35310, Neurospora crassa.

An “expression cassette” refers to a nucleic acid construct, which when introduced into a host cell, results in transcription and/or translation of a RNA or polypeptide, respectively.

In the case of expression of transgenes one of skill will recognize that the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “IspS polynucleotide sequence” or “IspS gene”.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. A “comparison window”, as used herein, includes reference to a segment of any one of the number of contiguous positions, e.g., 20 to 600, usually about 50 to about 200, more usually about 100 to about 150 in which a sequence may be compared to a reference sequence of the same number of contiguous positions after the two sequences are optimally aligned.

The term “substantial identity” in the context of polynucleotide or amino acid sequences means that a polynucleotide or polypeptide comprises a sequence that has at least 50% sequence identity to a reference sequence. Alternatively, percent identity can be any integer from 50% to 100%. Exemplary embodiments include at least: 55%, 57%, 60%, 65%, 70%, 75%, 80%, 85%, 90%, 95%, or 99% identity compared to a reference sequence using the programs described herein; preferably BLAST using standard parameters, as described below. Accordingly, IspS sequences of the invention include nucleic acid sequences that have substantial identity to the IspS coding regions of SEQ ID NO:1, SEQ ID NO:3, SEQ ID NO:4, or SEQ ID NO:6, or any one of SEQ ID NOs 8-11. As noted above, IspS polypeptide sequences of the invention include polypeptide sequences having substantial identify to SEQ ID NO:2, SEQ ID NO:5 or SEQ ID NO:7.

Polypeptides that are “substantially similar” share sequences as noted above except that residue positions that are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2 SSC, and 0.1% SDS at 55° C., 60° C., or 65° C. Such washes can be performed for 5, 15, 30, 60, 120, or more minutes.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. For example, an IspS polynucleotides, can also be identified by their ability to hybridize under stringency conditions (e.g., Tm ˜40° C.) to nucleic acid probes having the sequence of SEQ ID NO:1, SEQ ID NO:3, SEQ ID NO:4, or SEQ ID NO:6, or any one of SEQ ID NOs 8-11. Such an IspS nucleic acid sequence can have, e.g., about 25-30% base pair mismatches or less relative to the selected nucleic acid probe. SEQ ID NO:1 is an exemplary IspS polynucleotide sequence. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

The term “isolated”, when applied to a nucleic acid or protein, denotes that the nucleic acid or protein is essentially free of other cellular components with which it is associated in the natural state. It is preferably in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest.

As used herein, “mass-culturing” refers to growing large quantities of an Ascomycota, e.g., a yeast or a fungus, that have been modified to express an IspS gene. A “large quantity” is generally in the range of about 100 liters to about 1,500,000 liters, or more. In some embodiments, the organisms are cultured in large quantities in modular bioreactors, each having a capacity of about 1,000 to about 1,000,000 liters.

A “bioreactor” in the context of this invention is any enclosed large-capacity vessel in which Ascomycota microorganisms, e.g., yeast or fungus, are grown. A “large-capacity vessel” in the context of this invention can hold about 100 liters, often about 500 liters, or about 1,000 liters to about 1,000,000 liters, or more. The term encompasses fermentors used for anaerobic growth of organisms as well as vessels used for aerobic growth conditions.

As used herein, “harvesting” volatile isoprene hydrocarbons refers to capturing and sequestering such hydrocarbons in a closed or contained environment.

### IspS, MVA Pathway Nucleic Acid Sequences

The invention employs various routine recombinant nucleic acid techniques. Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel, et al., John Wiley and Sons, New York, 2008).

IspS nucleic acid and polypeptide sequences are known in the art. IspS genes have been isolated and sequenced from poplar and aspen (two related trees), and kudzu (a vine). The species involved and the sequences available in the NCBI database are given below by accession number, each of which is incorporated by reference:

Populus alba (white poplar) IspS mRNA for isoprene synthase; ACCESSION No AB198180;

Populus tremuloides (quaking aspen) isoprene synthase (IspS); ACCESSION No AY341431 (complete cds);

Populus alba x Populus tremula IspS mRNA; ACCESSION No AJ294819;

Populus nigra (Lombardy poplar) mRNA for isoprene synthase (IspS gene); ACCESSION No AM410988;

Pueraria montana var. lobata (kudzu vine) isoprene synthase (IspS); ACCESSION No AY316691 (complete cds.).

Examination of these IspS sequences reveals a high degree of nucleotide and amino acid sequence identities, for example, hybrid poplar and aspen cDNA sequences are 98% identical at the polypeptide and nucleotide level (see, e.g., Sharkey et al., Plant Physiol. 137:700-712, 1995). The aspen isoprene synthase nucleotide coding sequence is 65% identical to the kudzu gene, while the protein sequences (without the chloroplast transit peptide) are 57% identical.

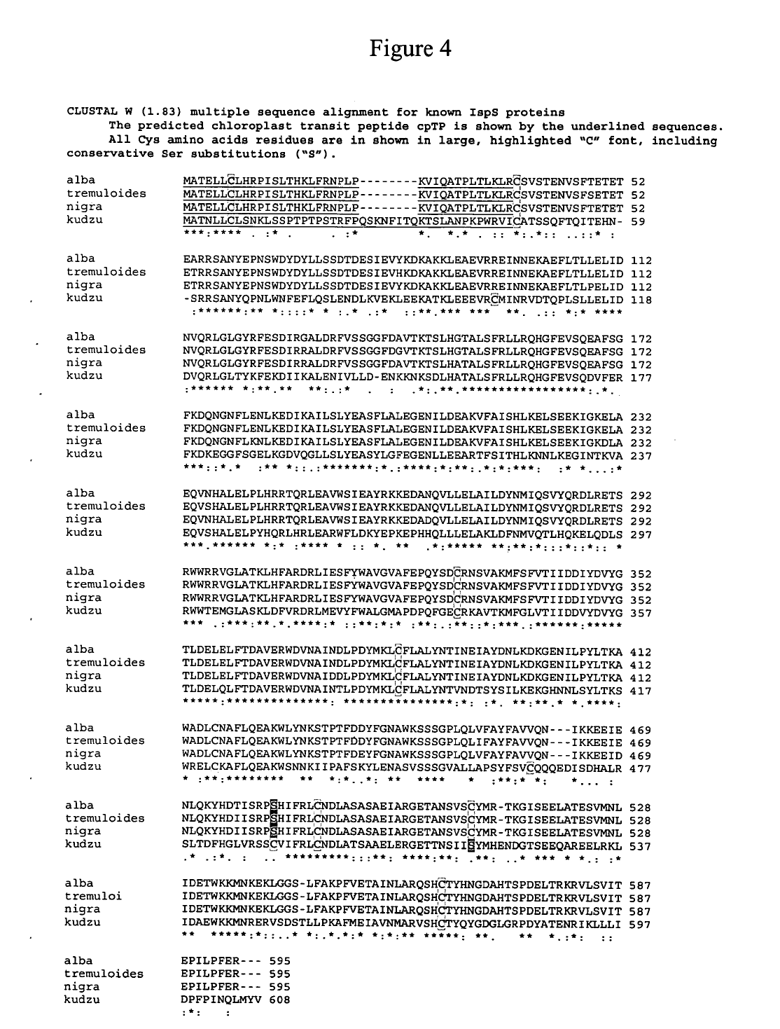

The poplar IspS protein has a high-density of conserved cysteine and histidine amino acids in the carboxy-terminal half of the protein. For example, considering the 591 amino acid sequence of the poplar IspS protein, cysteine moieties are found at positions 34, 326, 378, 413, 484, 505 and 559, i.e., six out of the seven cysteines are found in the lower 45% of the protein. Additional clustering of histidines in various positions of the C-terminal half of the protein is also observed. Cysteine and histidine amino acids are known to participate in proper folding and catalytic site structure of proteins and can be important components for enzyme activity. An alignment of four known IspS proteins showing the high conservation of Cys in the C-terminal part of the molecule is provided in FIG. 4. In one case, the kudzu protein has substituted an otherwise conserved Cys with Ser (Cys-509-Ser) of the alba or nigra or tremuloides sequence in the Clustal W alignment in FIG. 4. Serine is a highly conservative substitution for cysteine, as the only difference between the two amino acids is a —OH group in the place of the —SH group. In fact, examination of the four IspS sequences reveals the additional property of many conserved Serines in the C-terminal half of the protein. Accordingly, in some embodiments, a nucleic acid for use in the invention encodes an IspS polypeptide that comprises the carboxyl-terminal 45% of SEQ ID NO:2 and retains the catalytic activity in converting IPP or DMAPP to isoprene. Thus, in some embodiments, an IspS nucleic acid for use in the invention encodes a polypeptide that comprises from about amino acid residue 330 through the C-terminus of SEQ ID NO:2. In some embodiments, the IspS polypeptide encoded by the IspS nucleic acid comprises from about amino acid residue 300 through the C-terminus of SEQ ID NO:2 or SEQ ID NO:7. In some embodiments, the IspS sequence can additionally lack the last 10 or 15 C-terminal residues of SEQ ID NO:2, SEQ ID NO:5, or SEQ ID NO:7. As understood in the art, one of skill can also use the known sequences, e.g., an alignment such as the alignment shown in FIG. 4, to identity IspS proteins that retain activity but have amino acid residues that may be altered relative to the reference sequences shown in the alignment.

The transit peptide of the IspS protein includes, minimally, amino acids 1-37 for poplar and aspen and 1-45 for kudzu. On the basis of this analysis, the mature IspS protein begins with the amino acid sequence “CSVSTEN . . . ” for poplar and aspen IspS and “CATSSQ . . . ” for kudzu IspS. IspS nucleic acid sequences for use in the invention need not include sequences that encode a transit peptide and further, can omit additional N-terminal residues.

In some embodiments of the invention, a nucleic acid sequence that encodes a kudzu IspS polypeptide (e.g., SEQ ID NO:2) is used. In other embodiments, a nucleic acid sequence that encodes a poplar or aspen IspS polypeptide (e.g., SEQ ID NO:7) is used. The IspS polypeptides encoded by the nucleic acids employed in the methods of the invention have the catalytic activity of converting IPP or DMAPP to isoprene. Typically, the level of activity is equivalent to the activity exhibited by a poplar or aspen IspS polypeptide (e.g., SEQ ID NO:7) or a kudzu IspS polypeptide (e.g., SEQ ID NO:2 or 5).

In some embodiments of the invention, the activity of one or more MVA pathway enzymes is also increased to provide for enhanced production of volatile short chain hydrocarbons. Activity can be increased by various methodologies, e.g., altering substrate availability. In some embodiments, activity of an MVA pathway enzyme is increased by enhancing expression of the enzyme using recombinant expression techniques. Typically, MVA enzymes that are of interest to express are HMG CoA reductase and HMG CoA synthase. Genes encoding these enzymes are widely known. An HMG-CoA synthase and/or hMG-CoA reductase nucleic acid sequence can be introduced into an Ascomycota, e.g., a yeast or Neurospora, using techniques known in the art.

Isolation or generation of IspS and MVA pathway, e.g., HMG-CoA reducatase and HMG-CoA synthase polynucleotide sequences can be accomplished by a number of techniques. Cloning and expression of such technique will be addressed in the context of IspS genes. However, the same techniques can be used to isolate and express enzymes of the MVA pathway, including HMG-CoA synthase and/or HMG-CoA reductase. For instance, oligonucleotide probes based on the sequences disclosed here can be used to identify the desired polynucleotide in a cDNA or genomic DNA library from a desired plant species. Such a cDNA or genomic library can then be screened using a probe based upon the sequence of a cloned IspS gene, e.g., SEQ ID NO:1 or SEQ ID NO:6. Probes may be used to hybridize with genomic DNA or cDNA sequences to isolate homologous genes in the same or different plant species.

Alternatively, the nucleic acids of interest can be amplified from nucleic acid samples using amplification techniques. For instance, PCR may be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying an IspS gene from plant cells, e.g., poplar or another deciduous tree, can be generated from comparisons of the sequences provided herein. For a general overview of PCR see PCR Protocols: A Guide to Methods and Applications. (Innis, M, Gelfand, D., Sninsky, J. and White, T., eds.), Academic Press, San Diego (1990). An exemplary PCR for amplifying an IspS nucleic acid sequence is provided in the examples.

IspS nucleic acid sequences for use in the invention includes genes and gene products identified and characterized by techniques such as hybridization and/or sequence analysis using exemplary nucleic acid sequences, e.g., SEQ ID NO:1 or SEQ ID NO:6, and protein sequences, e.g., SEQ ID NO:2 or SEQ ID NO:7.

### Preparation of Recombinant Vectors

To use isolated sequences in the above techniques, recombinant DNA vectors suitable for transformation of Ascomycota cells, are prepared. Techniques for transformation are well known and described in the technical and scientific literature. For example, a DNA sequence encoding an IspS gene (described in further detail below), can be combined with transcriptional and other regulatory sequences which will direct the transcription of the sequence from the gene in the intended cells, e.g., yeast or filamentous fungi. In some embodiments, an expression vector that comprises an expression cassette that comprises the IspS gene further comprises a promoter operably linked to the IspS gene. In other embodiments, a promoter and/or other regulatory elements that direct transcription of the IspS gene are endogenous to the microorganism, e.g., yeast, and the expression cassette comprising the IspS gene is introduced, e.g., by homologous recombination, such that the heterologous IspS gene is operably linked to an endogenous promoter and is expression driven by the endogenous promoter.

Regulatory sequences include promoters, which may be either constitutive or inducible. In some embodiments, a promoter can be used to direct expression of IspS nucleic acids under the influence of changing environmental conditions. Examples of environmental conditions that may effect transcription by inducible promoters include anaerobic conditions, elevated temperature, or the presence of light. Promoters that are inducible upon exposure to chemicals reagents are also used to express IspS nucleic acids. Other useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element also can be, for example, a nitrate-inducible promoter, e.g., derived from the spinach nitrite reductase gene (Back et al., Plant Mol. Biol. 17:9 (1991)), or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)), or a light.

In yeast, a number of vectors containing constitutive or inducible promoters may be used. See for example, Current Protocols in Molecular Biology, Ausubel, supra; Grant, et al., 1987. A constitutive yeast promoter such as ADH or LEU2 or an inducible promoter such as GAL may be used (Cloning in Yeast, Ch. 3, R. Rothstein In: DNA Cloning Vol. 11, A Practical Approach, Ed. D M Glover, 1986, IRL Press, Wash., D.C.). Other examples of promoters suitable for use in yeast include CYC1, HIS3, PGK, PHO5, GAPDH, ADC1, TRP1, URA3, ENO, TPI (e.g., useful for expression in Saccharomyces) and AOX1 (e.g., useful for expression in Pichia). Alternatively, vectors may be used which promote integration of foreign DNA sequences into the yeast chromosome.

Promoters that can be used in non-yeast fungi, e.g., filamentous fungi such as Neurospora, include promoters from the β-tubulin gene, the grg-1 gene, invertase, and the like. Teachings on transforming filamentous fungi are reviewed in U.S. Pat. No. 5,741,665 and in U.S. Pat. No. 5,695,965. Further techniques as applied to Neurospora crassa are found, for example in Davis and de Serres, Methods Enzymol (1971) 17A: 79-143. Further teachings on transforming filamentous fungi are reviewed in U.S. Pat. No. 5,674,707. A transgenic Aspergillus according to the present invention can also be prepared by following, for example, the teachings of Turner G. 1994 (Vectors for genetic manipulation. In: Martinelli S. D., Kinghorn J. R.(Editors) Aspergillus: 50 years on. Progress in industrial microbiology vol 29. Elsevier Amsterdam 1994. pp. 641-666). Gene expression in filamentous fungi has additionally been reviewed in Punt et al. (2002) Trends Biotechnol 2002 May; 20(5):200-6, Archer & Peberdy Crit. Rev Biotechnol (1997) 17(4):273-306.

In some embodiments, promoters are identified by analyzing the 5′ sequences of a genomic clone corresponding to an IspS gene. Sequences characteristic of promoter sequences can be used to identify the promoter.

A promoter can be evaluated, e.g., by testing the ability of the promoter to drive expression in the Ascomycota cells of interest, yeast or Neurospora, in which it is desirable to introduce an IspS expression construct.

A vector comprising IspS nucleic acid sequences will typically comprise a marker gene that confers a selectable phenotype on the cell to which it is introduced. Such markers are known. For example, the marker may encode antibiotic resistance, such as resistance to kanamycin, G418, bleomycin, hygromycin, and the like.

Vectors and methods for the construction of vectors that are suitable for use in fungi, such as the filamentous fungi, include those that are described in: van den Hondel, C.A.M.J.J. (J. F. Peberdy, Ed., pp. 1-28, Cambridge University Press: Cambridge; or in: More Gene Manipulations in Fungi; J. W. Bennet & L. L. Lasure, Ed., pp. 396-428: Academic Press: San Diego, 1991). Examples of suitable yeast vectors are 2αM, pAG-1, YEp6, YEp13 or pEMBLYe23. Additional examples of expression systems and transformation of yeast and fungi, e.g., filamentous fungi can be found, e.g., in U.S. Patent Application Publication Nos. 2007/0196449 and 20060257923.

IspS nucleic acid sequences of the invention are expressed recombinantly in Ascomycota microorganisms, e.g., yeast, or filamentous fungi. As appreciated by one of skill in the art, expression constructs can be designed taking into account such properties as codon usage frequencies of the organism in which the IspS nucleic acid is to be expressed. Codon usage frequencies can be tabulated using known methods (see, e.g., Nakamura et al. Nucl. Acids Res. 28:292, 2000). Codon usage frequency tables, including those for yeast such as Saccharomyces cerevisiae and filamentous fungi such as Neurospora crassa or Aspergillus fumigatus are available in the art (e.g., from the Codon Usage Database at the internet site www.kazusa.or.jp/codon/.

Cell transformation methods and selectable markers for Ascomycota such as yeast and filamentous fungi, e.g., Neurospora are well known in the art, e.g., in references that review yeast and filamentous fungus expression systems. Transformation techniques include, e.g., electroporation, liposome-mediated transfection, the use of magnetic particles, and the like.

The techniques described herein for obtaining and expressing IspS nucleic acid sequences in Ascomycota can also be employed to express nucleic acid sequences that encode MVA pathway enzymes, e.g., HMG-CoA reductase or HMG-CoA synthase.

Microorganisms that can be Targeted

IspS can be expressed in any number of Ascomycota where it is desirable to produce isoprene. Transformed Ascomycota cells that express a heterologous IspS gene are grown under mass culture conditions for the production of hydrocarbons, e.g., to be used as a fuel source or as feedstock in synthetic chemistry. The transformed organisms are growth in bioreactors or fermentors that provide an enclosed environment to contain the hydrocarbons. In typical embodiments for mass culture, the transformed cells are grown in enclosed reactors in quantities of at least about 500 liters, often of at least about 1000 liters or greater, and in some embodiments in quantities of about 1,000,000 liters or more.

In some embodiments, IspS is expressed in yeast. Synthesis of heterologous proteins in yeast is well known and described in the literature. For example, Methods in Yeast Genetics, Sherman, F., et al., Cold Spring Harbor Laboratory, (1982) describes various methods available to express proteins in yeast. Exemplary yeast cells include any species of Pichia, Saccharomyces, Schizosaccharomyces, Kluyveromyces, Candida, Pachysolen, Hansenula, or Schwanniomyces, including Pichia pastoris, Saccharomyces cerevisiae, or Schizosaccharomyces pombe.

In some embodiments, IspS is expressed in a filamentous fungus. Any filamentous fungus can be employed. Filamentous fungi that are useful for industrial application are known (see, e.g., Applied Molecular Genetics of Filamentous Fungi, Kinghorn & Turner, eds., Chapman & Hall, New York.1992; Molecular Industrial Mycology: Systems and Applications for Filamentous Fungi, Leong & Berka, eds., Marcel Dekker, New York, 1991). For example, filamentous fungi are widely utilized to produce organic acids (Bizukojc & Ledakowicz, Process Biochemistry 39:2261-2268, 2004); and proteins (Wang, et al., Biotechnology Advances 23:115-129, 2003).

Filamentous fungi in which IspS can be expressed includes any member belonging to the genera Neurospora, Thermomyces, Acremonium, Aspergillus, Penicillium, Mucor, and Trichoderma, and Rhizopus. Other filamentous fungi include Cephalosporium, Achlya, Podospora, Mucor, Cochliobolus, and Pyricularia. (See, e.g., U.S. Pat. No. 5,679,543 and Stahl and Tudzynski, Eds., Molecular Biology in Filamentous Fungi, John Wiley & Sons, 1992.)

Methods of mass-culturing yeast and fungi are known. For example, yeast and fungi can be grown to high cell densities in a bioreactor or fermentor supplied with growth media containing sugars, bioorganic polymers (starch, cellulose, hemicellulose), biomass, or cellular metabolites. Aerobic catabolism of bioorganic substrate in bioreactors for the generation of isoprene is one viable metabolic approach in this process. Anaerobic catabolism of bioorganic substrate in a closed bioreactor (fermentor) for the generation of isoprene is another viable approach in this endeavor. A regimen of alternating aerobic and anaerobic conditions during growth of yeast/fungi typically enhances the efficiency of bioorganic substrate catabolism and improves yields of isoprene production. Examples of mass-cultivation techniques for yeast/fungi are additionally available in references that describe production of various compounds, including antibiotic and pharmaceuticals using yeast and filamentous fungi. For example, U.S. Patent Application Publication No. 20070292927 describes production of four-carbon alcohols using known fermentation techniques. In another example, production of coconut aroma by fungi cultivation in solid-state fermentation offers an additional example of cultivation of microorganisms for production of bioproducts. (e.g., Applied Biochemistry and Biotechnology Vol 99:747-752, 2002).

Conditions for growing IspS-expressing Ascomycota, e.g., yeast or filamentous fungi, for the exemplary purposes illustrated above are known in the art (see, e.g., the exemplary references cited herein). Volatile isoprene hydrocarbons produced by the modified microorganisms can be harvested using known techniques. Isoprene hydrocarbons are not miscible in water and they rise to and float at the surface of the microorganism growth medium. They are siphoned off from the surface and sequestered in suitable containers. In addition, and depending on the prevailing temperature during the mass cultivation of the microorganisms, isoprene can exist in vapor form above the water medium in the bioreactor container (isoprene boiling temperature T=34° C.). Isoprene vapor is piped off the bioreactor container and condensed into liquid fuel form upon cooling or low-level compression.

### Examples

The examples described herein are provided by way of illustration only and not by way of limitation. Those of skill in the art will readily recognize a variety of non-critical parameters that could be changed or modified to yield essentially similar results.

### Example 1

### Design and Expression of Novel IspS Genes for Isoprene Hydrocarbon Production in Yeast and Fungi

In Fungi/Ascomycota (yeast/fungi) cells, the nuclear-encoded and cytosol-localized enzyme of the mevalonic acid pathway can be directed to produce isoprene following suitable genetic heterologous modification of the microorganism to express the isoprene synthase gene. Heterologous expression of the IspS gene in Fungi/Ascomycota cells confers the ability to produce volatile isoprene hydrocarbons.

A codon-adjusted synthetic DNA construct was generated based on the known nuclear-encoded “isoprene synthase” IspS protein sequence of Pueraria Montana (kudzu). This amino acid sequence (SEQ ID NO:2) was used as a template. Features of the yeast and fungi genes include: (1) Codon usage is different from that of the kudzu and specifically selected to fit the codon usage of yeast and fungi, and (2) the kudzu chloroplast-targeting sequence of the protein was omitted from the design of the new IspS genes.

The yeast IspS sequence (SEQ ID NO:3) was designed to encode for the isoprene synthase protein (SEQ ID NO:5). Codon usage adjustments for gene expression in yeast were made on the basis of the codon usage table for yeast from the Codon Usage Database developed and maintained by Yasukazu Nakamura at The First Laboratory for Plant Gene Research, Kazusa DNA Research Institute, Japan).

A fungi IspS gene sequence (SEQ ID NO:4) was also generated with codon usage adjustments based on the codon usage table for Neuropora. This gene encodes an isoprene synthase as shown in (SEQ ID NO:5).

As understood in the art, alternative codon-optimized genes may also be designed that vary somewhat in nucleotide sequence, but encode the same amino acid sequence. For example, SEQ ID NO:8-11 provide alternative codon-optimized genes for expressing isoprene synthase (SEQ ID NO:5) in yeast.

Transgenic yeast and Neurospora are generated that express the codon-optimized recombinant isoprene synthase genes using expression techniques. Such transgenic organisms can be used to produce isoprene and other short-chain volatile isoprenes.

Strains used as Culture Conditions:

Escherichia coli strain DH5α F′^{e} [F′/endA1 hsdR17 (rₖ⁻ mₖ⁺) supE44 thi-1 recA1 gyrA (Nal′) relA1 Δ(lacZYA-argF)ᵤ₁₆₉: (m80ΔlacZM15)] (Gibco BRL, Life Technologies Inc, Rockville, Md.) was used to amplify plasmid DNA using standard procedures (e.g., Ausubel, supra, 2008). Saccharomyces cerevisiae 20B-12 (MATαpep4-3 trp1) and S. cerevisiae AH22 (MATα leu2-3 leu2-112 his4-519 can1) (ATCC, Manassas, Va.) were used as the recipient strains for plasmids containing S. cerevisiae codon-optimized kudzu vine IspS plasmids (pSckIspStg or pSckIspSlg) and plasmids containing the original sequence of the kudzu vine IspS (pkIspStg or pkIspSlg), respectively. E. coli was cultured on LB medium (Luria-Bertani; 1% tryptone, 0.5% yeast extract, 1% sodium chloride) or LB containing appropriate antibiotics for competent cell preparation and plasmid isolation. Saccharomyces cerevisiae 20B-12 and S. cerevisiae AH22 strains were grown on YPD medium (1% yeast extract, 2% peptone, 2% glucose). YNBD medium (0.67% yeast nitrogen base without amino acids, 2% glucose; for S. cerevisiae 20B-12) or YNBD+0.01% histidine (for S. cerevisiae AH22) as selective media for yeast transformants (Lopes et al., Gene 79:199-206 1989).

Construction of Plasmids Containing S. cerevisiae Codon-Optimized IspS or Original Sequence of Kudzu Vine IspS.

pNGVF served as the plasmid backbone. The pNGVF was constructed using pAPNGFPVFNB and pGFPV (Hong and Linz, Appl Environ Microbiol 74:6385-6396, 2008). The pAPNGFPVFNB was digested with NotI and AscI, and the 3.7 kb fragment from the pAPNGFPVFNB replaced 2.8 kb fragment in pGFPV digested with the same enzymes, resulting in pNGVF. A 0.65 kb GPD promoter was generated by PCR with Pfu DNA polymerase (Stratagene, La Jolla, Calif.), appropriate primers, and p424 GPD (ATCC, Manassas, Va.) as a template using standard procedures (e.g., Ausubel et al. supra, 2008) (see Table 1 for primer sequences). PCR was performed in a PTC-200 Peltier Thermal Cycler (Bio-Rad, Hercules, Calif.). The reaction conditions were as follows: 94° C. for 5 min followed by 35 cycles of 94° C. for 1 min, annealing for 1 min (see Table 1 for annealing temperatures), and extension at 72° C. (time dependent on PCR fragment size: 2 min/1 kb). The reaction was completed with a final extension at 72° C. for 10 min. The PCR fragment was digested with NotI and SgfI and cloned into pNGVF cut with the same enzymes, resulting in pGVF. A 0.8 kb TRP1d fragment was generated by PCR with Pfu DNA polymerase, appropriate primers, and p424 GPD (ATCC, Manassas, Va.) as a template using standard procedures (e.g., Ausubel et al., supra, 2008) (see Table 1 for primers). Similar PCR conditions were used as for pGVF (Table 1). The PCR fragment digested with PacI and NotI was cloned into pGVF cut with the same enzymes, resulting in pTGVF. To subclone a CYC1 terminator fragment, the 0.26 kb fragment was generated by PCR with Pfu DNA polymerase, appropriate primers, and p424 GPD (ATCC, Manassas, Va.) as a template using standard procedures (for primers, see Table 1). Similar PCR conditions were used as for pGVF (Table 1). The PCR fragment was cloned into the SmaI site of pUC18, resulting in pUCCYC1t. To subclone rDNA fragments, the 0.67 kb and 0.59 kb fragments were generated by PCR with Pfu DNA polymerase, appropriate primers, and genomic DNA as a template using standard procedures (e.g., Ausubel et al. 2008) (for primers, see Table 1). Genomic DNA isolation from S. cerevisiae 20B-12 was done according to Philippsen et al., Methods Enzymol 194:169-182, 1991 with minor modifications. Similar PCR conditions were employed as for the construction of pGVF (Table 1). The 0.67 kb rDNA fragment was subcloned into pUCCYC1t, which was first digested with AsiSI and AscI, resulting in pUCCD. DNA fragments containing the CYC1 terminator and the rDNA were then subcloned from pUCCD into pTGVF, cut with FseI and AscI, resulting in pTGVCD. The 0.59 kb rDNA fragment was subcloned into pTGVCD, which was first digested with SbfI and PacI, resulting in pDTGVCD.

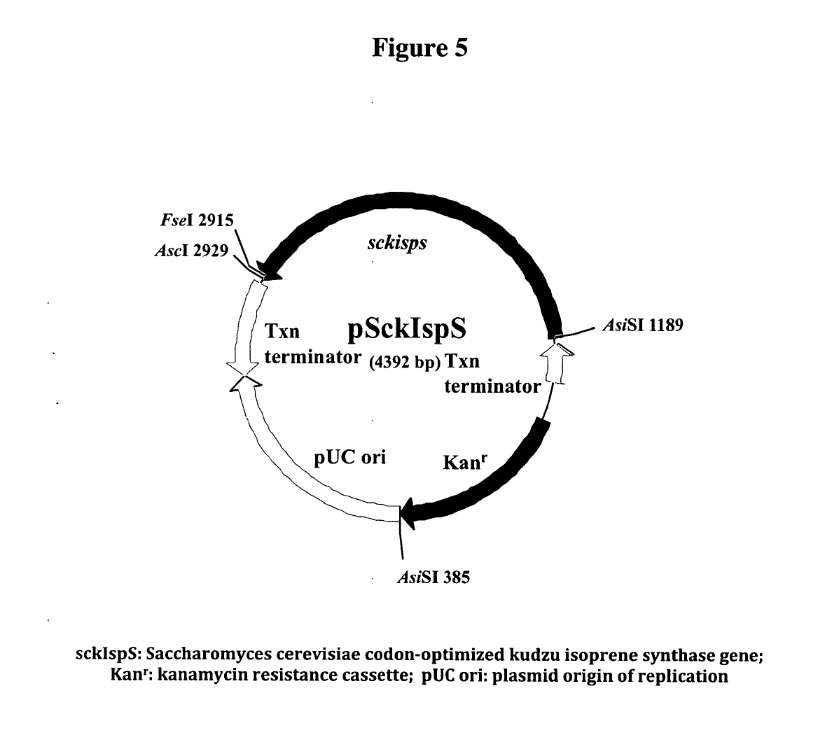

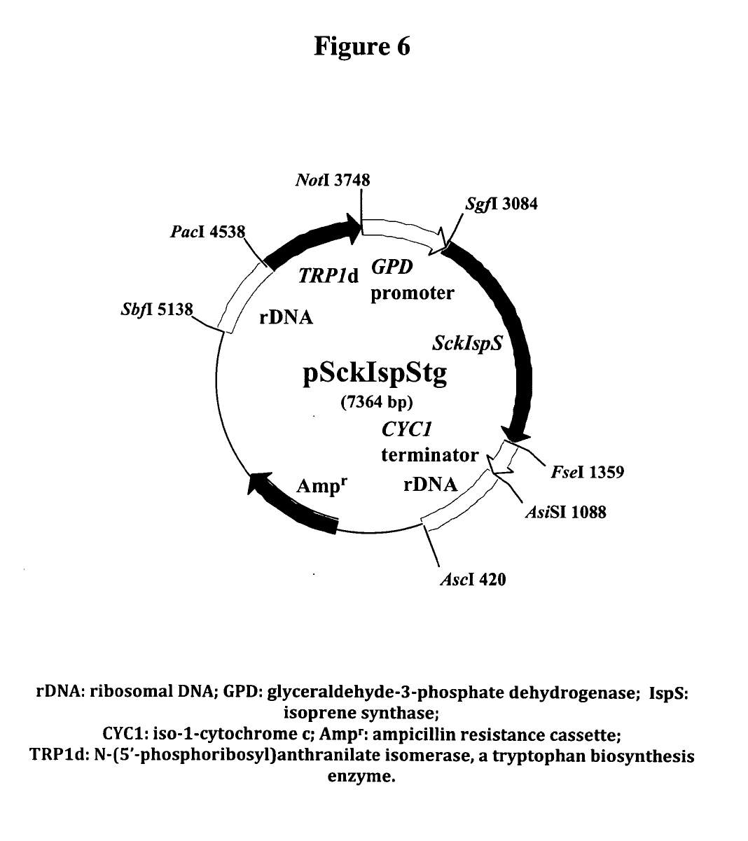

A 1.7 kb S. cerevisiae codon-optimized kudzu vine “Isoprene Synthase” SckIspS gene (cDNA) and the original sequence of the kudzu vine IspS gene (cDNA) were synthesized and subcloned to generate plasmid pSckIspS (FIG. 5) and pkudzu-isps, respectively. DNA fragments containing the CYC1 terminator and the 0.67 kb rDNA were then subcloned from pUCCD into pSckIspS, which was first digested with FseI and AscI, resulting in pSckIspSCD. Finally, DNA fragments containing the SckIspS and the CYC1 terminator were subcloned from pSckIspSCD into pDTGVCD, which was first digested with AsiSI(=SgfI), resulting in pSckIspStg (FIG. 6). To construct the plasmid containing 1.7 kb Kudzu vine IspS fragment, the fragment was generated by PCR with Pfu DNA polymerase, appropriate primers, and pkudzu-isps as a template using standard procedures (for primers, see Table 1). Similar PCR conditions were used as for pGVF (Table 1). The 1.7 kb Kudzu vine IspS fragment was subcloned into the SmaI site of pUC18, resulting in pUCkIspS. DNA fragments containing the CYC1 terminator and the 0.67 kb rDNA were then subcloned from pUCCD into pUCkIspS cut with FseI and AscI, resulting in pUCkIspSCD. Finally, DNA fragments containing the kIspS and the CYC1 terminator were subcloned from pUCkIspSCD into pDTGVCD, cut with AsiSI(=SgfI), resulting in pkIspStg.

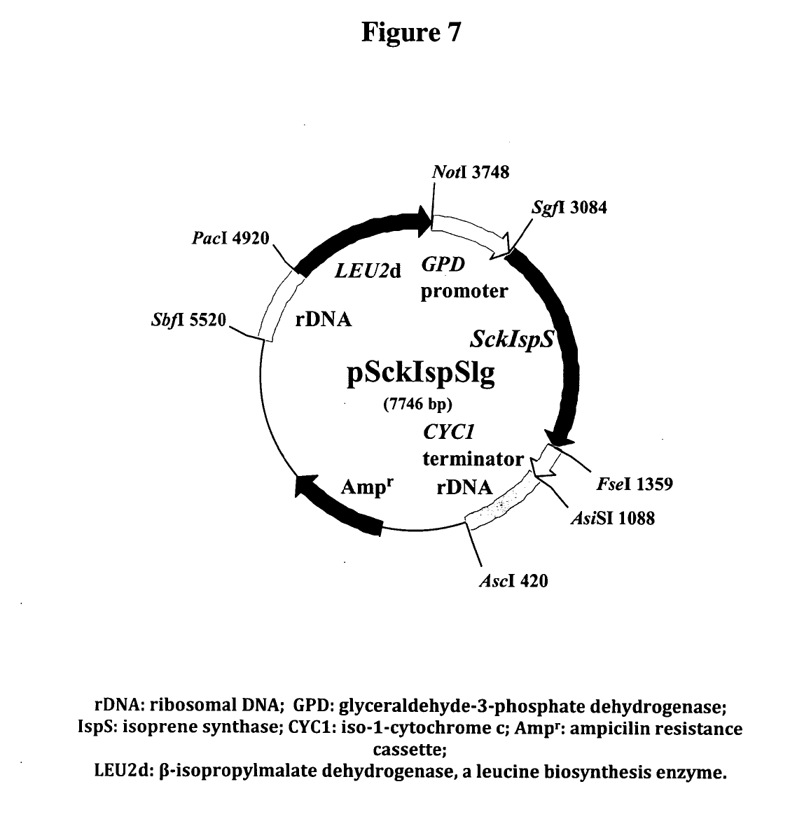

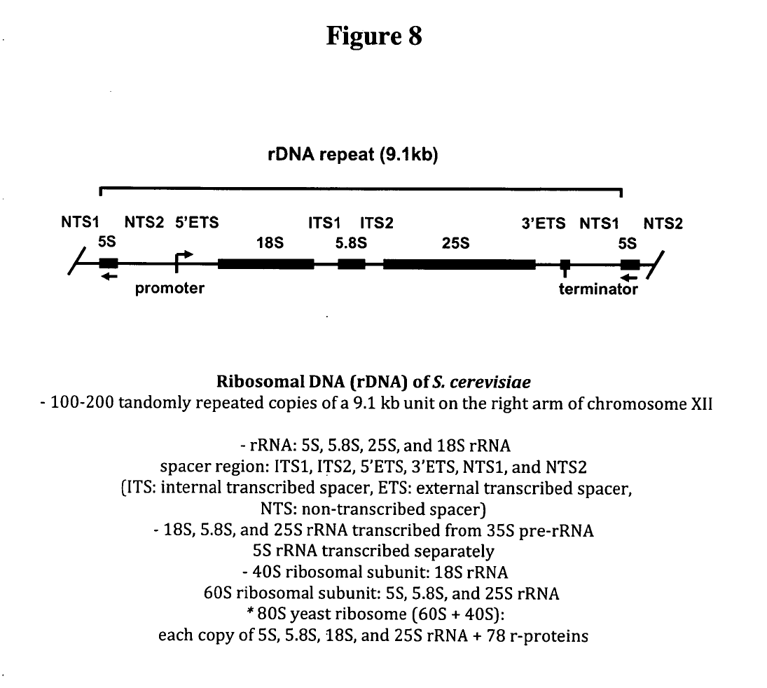

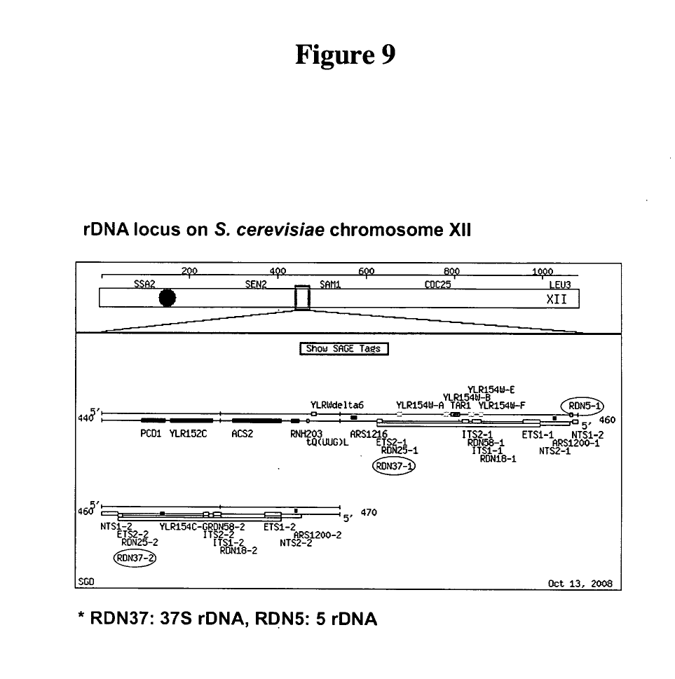

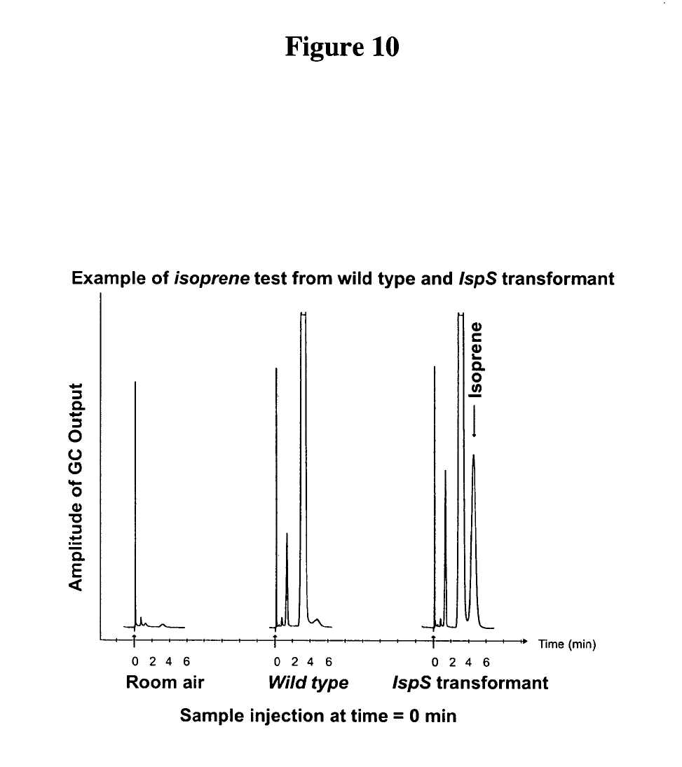

To construct plasmids pSckIspSlg (FIG. 7) and pkIspSlg, containing the LEU2d fragment, the TRP1d fragment was replaced by the LEU2d fragment.

Saccharomyces cerevisiae Transformations with Plasmid DNA.

For yeast transformation, plasmids were digested with restriction enzymes SbfI and AscI and the DNA fragments, containing the selectable markers (TRP1d or LEU2d), IspS gene, and rDNA regions were agarose gel-purified. Yeast transformation was performed by the lithium acetate method with minor modifications (Ito et al., J Bacteriol. 153:163-168, 1983) and transformants were selected.

### Example 2

### Growth of Saccharomyces cerevisiae Transformants and Isoprene Detection

For measurements of isoprene production, transformed yeast cells were first grown on agar plates. A small aliquot of the resultant colonies was inoculated in a starter liquid culture, followed by a small volume (1%) inoculation in an Erlenmeyer flask (50 ml capacity). Liquid cultures were grown in the presence of YPD media (tryptophan or leucine prototrophy) at 30° C. upon shaking in a rotary shaker to 150 rpm. Erlenmeyer flasks (50 ml capacity) were used for growth of the cells in a 20 ml volume, covered with Styrofoam stopper during cell growth, sealed by a silicon stopper for overnight incubation, prior to sampling of the gaseous headspace. Saccharomyces cerevisiae cultures in the early exponential growth phase were used for isoprene gas measurement experiments, as follows. One (1) ml of the headspace gas-phase was withdrawn by a gastight syringe and analyzed on a Shimadzu 8A GC equipped with a Porapak N 80/100 column and a flame-ionization detector. The isoprene peak (FIG. 10), appearing at about 5 min elution time, was identified by co-elution with a suitable isoprene standard. This example thus illustrates that Saccharomyces cerevisiae can be engineered to produce isoprene.

TABLE 1
Primer sequences used in plasmid construction and analysis

| | | Restriction | Annealing |
|---|---|---|---|
| Primer^{a} | Sequence^{b} | Enzyme Site | Temp (° C.) |
| GPD promoter- F | 5′ TGCGCGGCCGCTCGAGTTTATCA- | NotI | 55 |
| | -TTATCAATACT 3′ | | |
| GPD promoter- R | 5′ CTAGCGATCGCTTATTCGAAACT- | SgfI | 55 |
| | -AAGTTCTTGGT 3′ | | |
| TRP1d- F | 5′ GGATTAATTAAAGCACGTGAGT- | PacI | 57 |
| | -ATACCTGATT 3′ | | |
| TRP1d- R | 5′ TTAGCGGCCGCTTTCAAAACGCC- | NotI | 57 |
| | -TGCTGGCAAGTGCACAAACAATA- | | |
| | -C 3′ | | |
| CYC1 terminator- F | 5′CCCGGCCGGCCTTTGTCGATATC- | FseI | 50 |
| | -ATGTAATTAGTTATGT 3′ | | |
| CYC1 terminator- R | 5′ CGGGCGATCGCAAGCATGCAAA- | AsiSI | 50 |
| | -TTAAAGCCTTCGAG 3′ | | |
| rDNA1- F | 5′ GTACCTGCAGGATGAGAGTAGC- | SbfI | 50 |
| | -AAACG 3′ | | |
| rDNA1- R | 5′ TGATTAATTAACCGGGTAACCCA- | PacI | 50 |
| | -GTTC 3′ | | |
| rDNA2- F | 5′ GTTGCGATCGCCACCTGTCACTT- | AsiSI | 50 |
| | -TGGA 3′ | | |
| rDNA2- R | 5′ TTAGGCGCGCCCAGGTTCCACCA- | AscI | 50 |
| | -AACA 3′ | | |
| LEU2d- F | 5′ GGATTAATTAAATATATATTTCA- | PacI | 50 |
| | -AGGATA 3′ | | |
| LEU2d- R | 5′ GAAGCGGCCGCATAAAGTTTCT- | NotI | 50 |
| | -GTACAAATATCATAAAAAA 3′ | | |
| kIspS- F | 5′ CAGCGATCGCACACACATAAAT- | SgfI | 45 |
| | -AAACAAAATGCCATGGATT 3′ | | |
| kIspS- R | 5′ TAGGCGCGCCTGTCATGGATGG- | AscI, FseI | 45 |
| | -CCGGCCTTACACGTACATTAGTT 3′ | | |

^{a}F represents forward primers and R represents reverse primers.
^{b}Underlined sequences show the position of the restriction enzyme sites.

All publications, accession numbers, and patent applications cited in this specification are herein incorporated by reference as if each individual publication or patent application were specifically and individually indicated to be incorporated by reference.

### Exemplary IspS sequences

SEQ ID NO:1 Pueraria Montana cDNA sequence encoding isoprene synthase, available through GenBank Accession No. AY316691

| | |
|---|---|
| 1 | atggcaacca accttttatg cttgtctaat aaattatcgt cccccacacc aacaccaagt |
| 61 | actagatttc cacaaagtaa gaacttcatc acacaaaaaa catctcttgc caatcccaaa |
| 121 | ccttggcgag ttatttgtgc tacgagctct caatttaccc aaataacaga acataatagt |
| 181 | cggcgttcag ctaattacca gccaaacctc tggaattttg aatttctgca gtctctggaa |
| 241 | aatgacctta aggtggaaaa actagaagag aaggcaacaa agctagagga ggaggtacga |
| 301 | tgcatgatca acagagtaga cacacaacca ttaagcttac tagaattgat cgacgatgtc |
| 361 | cagcgtctag gattgaccta caagtttgag aaggacataa tcaaagccct tgagaatatt |
| 421 | gttttgctgg atgagaataa gaaaaataaa agtgacctcc atgctactgc tctcagcttc |
| 481 | cgtttactta gacaacatgg ctttgaggtt tcccaagatg tgtttgagag atttaaggac |
| 541 | aaggagggag gtttcagtgg tgaacttaaa ggtgatgtgc aagggttgct gagtctatat |
| 601 | gaagcatcct atcttggctt tgagggagaa aatctcttgg aggaggcaag gacattttca |
| 661 | ataacacatc tcaagaacaa cctaaaagaa ggaataaaca ccaaagtggc agaacaagtt |
| 721 | agtcatgcac tggaacttcc ctatcatcaa agattgcata gactagaagc acgatggttc |
| 781 | cttgacaaat atgaaccaaa ggaaccccac catcagttac tactcgagct tgcaaagcta |
| 841 | gatttcaata tggtgcaaac attgcaccag aaagaactgc aagacctgtc aaggtggtgg |
| 901 | acggagatgg ggctagcaag caagctagac tttgtccgag acagattaat ggaagtgtat |
| 961 | ttttgggcgt tgggaatggc acctgatcct caattcggtg aatgtcgtaa agctgtcact |
| 1021 | aaaatgtttg gattggtcac catcatcgat gatgtatatg acgtttatgg tactttggat |
| 1081 | gagctacaac tcttcactga tgctgttgag agatgggacg tgaatgccat aaacacactt |
| 1141 | ccagactaca tgaagttgtg cttcctagca ctttataaca ccgtcaatga cacgtcttat |
| 1201 | agcatcctta aagaaaaagg acacaacaac ctttcctatt tgacaaaatc ttggcgtgag |
| 1261 | ttatgcaaag cattccttca agaagcaaaa tggtcgaaca acaaaatcat tccagcattt |
| 1321 | agcaagtacc tggaaaatgc atcggtgtcc tcctccggtg tggctttgct tgctccttcc |
| 1381 | tacttctcag tgtgccaaca acaagaagat atctcagacc atgctcttcg ttctttaact |
| 1441 | gatttccatg gccttgtgcg ctcctcatgc gtcattttca gactctgcaa tgatttggct |
| 1501 | acctcagcgg ctgagctaga gaggggtgag acgacaaatt caataatatc ttatatgcat |
| 1561 | gagaatgacg gcacttctga agagcaagca cgtgaggagt tgagaaaatt gatcgatgca |
| 1621 | gagtggaaga agatgaaccg agagcgagtt tcagattcta cactactccc aaaagctttt |
| 1681 | atggaaatag ctgttaacat ggctcgagtt tcgcattgca cataccaata tggagacgga |
| 1741 | cttggaaggc cagactacgc cacagagaat agaatcaagt tgctacttat agaccccttt |
| 1801 | ccaatcaatc aactaatgta cgtgtaa |

SEQ ID NO:2 Pueraria Montana polypeptide sequence for isoprene synthase (from Accession No. AY316691) The transit peptide is underlined and includes the starting methionine.

MATNLLCLSNKLSSPTPTPSTRFPQSKNFITQKTSLANPKPWRVICATSS
QFTQITEHNSRRSANYQPNLWNFEFLQSLENDLKVEKLEEKATKLEEEVR
CMINRVDTQPLSLLELIDDVQRLGLTYKFEKDIIKALENIVLLDENKKNK
SDLHATALSFRLLRQHGFEVSQDVFERFKDKEGGFSGELKGDVQGLLSLY
EASYLGFEGENLLEEARTFSITHLKNNLKEGINTKVAEQVSHALELPYHQ
RLHRLEARWFLDKYEPKEPHHQLLLELAKLDFNMVQTLHQKELQDLSRWW
TEMGLASKLDFVRDRLMEVYFWALGMAPDPQFGECRKAVTKMFGLVTIID
DVYDVYGTLDELQLFTDAVERWDVNAINTLPDYMKLCFLALYNTVNDTSY
SILKEKGHNNLSYLTKSWRELCKAFLQEAKWSNNKIIPAFSKYLENASVS
SSGVALLAPSYFSVCQQQEDISDHALRSLTDFHGLVRSSCVIFRLCNDLA
TSAAELERGETTNSIISYMHENDGTSEEQAREELRKLIDAEWKKMNRERV
SDSTLLPKAFMEIAVNMARVSHCTYQYGDGLGRPDYATENRIKLLLIDPF
PINQLMYV

SEQ ID NO:3 Exemplary nucleotide sequence for yeast codon-optimized IspS cDNA;

catatg: Nde I restriction site for cloning

tctaga: Xba I restriction site for cloning

ATC, underlined: First encoded Isoleucine amino acid

TAA, underlined: Stop codon

catatgATCTGTGCTACATCATCTCAGTTTACCCAAATTACCGAACACAA
TTCCAGAAGAAGTGCTAATTATCAACCGAACCTATGGAATTTTGAATTTT
TACAATCACTGGAGAACGATTTAAAAGTTGAAAAGTTAGAGGAAAAGGCT
ACCAAACTTGAGGAAGAAGTTAGATGTATGATTAATAGGGTTGACACTCA
ACCTTTATCACTATTGGAATTAATTGATGATGTACAACGTCTGGGTTTGA
CCTATAAATTCGAAAAAGACATAATAAAGGCTCTAGAAAATATAGTCTTG
TTAGACGAAAACAAAAAGAATAAAAGCGACCTACATGCGACAGCATTATC
TTTTAGACTTTTAAGACAACATGGCTTTGAAGTATCCCAAGATGTATTCG
AAAGATTTAAAGATAAAGAGGGAGGTTTTAGCGGTGAATTAAAAGGTGAT
GTGCAAGGATTATTGAGTTTGTATGAAGCCTCCTATTTAGGATTCGAGGG
TGAGAACCTATTAGAAGAAGCTCGTACATTTTCCATTACTCATTTGAAAA
ACAATTTAAAGGAAGGTATAAACACCAAAGTTGCTGAACAAGTTTCGCAT
GCGCTGGAATTACCATATCACCAGAGACTACATAGGCTTGAAGCTAGATG
GTTCCTTGATAAGTACGAACCTAAGGAGCCACATCACCAATTGTTACTAG
AATTAGCCAAACTGGATTTCAATATGGTACAGACCCTTCATCAAAAAGAG
TTGCAAGATTTGTCAAGATGGTGGACCGAGATGGGCCTAGCCAGTAAATT
GGATTTTGTTAGAGATAGATTAATGGAAGTTTATTTCTGGGCATTAGGTA
TGGCACCAGATCCACAGTTTGGGGAATGTCGTAAAGCTGTCACAAAAATG
TTTGGTTTGGTTACAATCATAGACGACGTCTATGATGTATATGGTACTCT
AGACGAGTTGCAATTATTCACTGATGCTGTTGAAAGATGGGACGTTAATG
CAATTAATACTCTACCTGATTATATGAAGTTGTGTTTTCTAGCATTGTAT
AATACGGTAAACGATACATCATATTCTATTTTGAAAGAAAAGGGTCATAA
CAATCTTTCTTACTTGACAAAGTCCTGGAGAGAACTATGTAAGGCCTTCC
TACAAGAGGCAAAATGGTCCAATAATAAAATTATACCAGCATTTTCGAAA
TACTTAGAGAATGCAAGCGTATCCTCAAGTGGTGTTGCGTTGTTAGCTCC
AAGTTATTTTAGTGTTTGTCAACAACAAGAAGACATCTCCGACCATGCAC
TTCGTTCATTGACGGACTTTCATGGGCTTGTGAGATCATCCTGCGTGATC
TTCAGGCTTTGCAACGATTTAGCTACCTCTGCTGCTGAATTAGAACGTGG
TGAGACGACCAATAGCATTATATCTTACATGCATGAGAATGACGGGACAT
CCGAGGAGCAGGCAAGAGAAGAATTACGTAAGTTGATCGACGCCGAGTGG
AAAAAGATGAATCGTGAAAGAGTCAGCGACTCCACACTGCTTCCAAAAGC
CTTCATGGAGATCGCTGTTAACATGGCCAGAGTTAGTCATTGTACTTATC
AGTATGGCGATGGATTGGGTAGACCGGATTATGCAACTGAGAATAGAATC
AAACTTTTATTGATCGATCCGTTTCCCATTAACCAGCTAATGTATGTATA
Atctaga

SEQ ID NO:4 Exemplary nucleotide sequence for Neurospora crassa codon-optimized IspS cDNA

catatg: Nde I restriction site for cloning

tctaga: Xba I restriction site for cloning

ATT, underlined: First encoded Isoleucine amino acid

TAA, underlined: Stop codon

catatgATTTGTGCCACCTCTAGCCAGTTTACTCAGATTACCGAGCATAA
CTCTCGTCGGTCCGCCAACTACCAGCCGAACCTGTGGAACTTCGAGTTTC
TCCAAAGCCTCGAGAACGACCTTAAAGTCGAGAAGCTGGAGGAAAAGGCC
ACCAAGTTGGAAGAAGAGGTTCGGTGTATGATCAACCGCGTCGATACACA
GCCCCTGTCTCTGCTCGAGCTCATCGACGACGTCCAACGCCTCGGCCTTA
CGTACAAGTTCGAAAAGGATATCATCAAGGCACTGGAGAACATCGTGCTT
CTCGACGAAAACAAGAAGAACAAATCTGACCTGCACGCAACCGCCTTGTC
CTTCCGCTTGTTGCGCCAGCACGGTTTCGAGGTGTCGCAGGACGTGTTTG
AGCGCTTCAAGGACAAGGAGGGTGGTTTCTCTGGCGAGCTGAAGGGAGAT
GTTCAAGGCCTTTTGAGCCTCTACGAGGCCAGCTACCTCGGTTTCGAAGG
AGAGAACCTCCTTGAAGAAGCTCGTACGTTCTCCATCACCCATCTCAAGA
ACAACCTTAAGGAAGGTATCAACACGAAGGTGGCAGAGCAAGTTTCCCAC
GCTCTCGAGCTGCCTTACCACCAGCGCCTGCATCGCTTGGAGGCCCGCTG
GTTCCTGGATAAGTACGAACCAAAGGAGCCCCACCATCAGCTGCTCCTCG
AGCTGGCTAAACTTGACTTCAATATGGTTCAGACTCTGCACCAAAAGGAA
CTGCAAGACCTCTCGCGTTGGTGGACCGAAATGGGTCTTGCAAGTAAGCT
GGACTTTGTGCGTGACCGCCTTATGGAGGTGTACTTCTGGGCTCTGGGCA
TGGCTCCAGACCCCCAGTTCGGTGAGTGCCGCAAGGCTGTGACGAAGATG
TTTGGTCTGGTTACCATTATTGACGACGTGTACGACGTTTATGGAACGCT
CGACGAGCTTCAGCTTTTCACAGACGCCGTGGAGCGGTGGGACGTTAATG
CCATCAACACCCTGCCCGATTACATGAAGCTGTGCTTTTTGGCCCTTTAC
AACACAGTGAATGATACGTCGTACTCTATTCTCAAAGAGAAGGGTCACAA
CAACCTCTCTTACTTGACTAAGTCGTGGCGTGAACTGTGCAAGGCCTTCC
TTCAGGAGGCGAAATGGAGCAATAATAAGATCATTCCCGCCTTCTCCAAG
TATCTGGAAAACGCCAGTGTGTCCAGCTCGGGCGTCGCCTTGCTGGCCCC
CAGTTATTTCTCTGTTTGCCAGCAACAGGAGGATATCTCCGACCATGCCC
TGCGTTCCCTGACCGATTTCCATGGACTGGTCCGCTCCTCCTGCGTTATT
TTCCGTCTGTGTAACGATCTGGCCACTAGCGCTGCGGAGCTGGAACGCGG
AGAGACGACCAACTCCATTATCTCCTACATGCACGAGAACGATGGCACCA
GTGAAGAGCAGGCCCGCGAGGAGCTTCGCAAACTGATCGACGCTGAGTGG
AAGAAGATGAATCGTGAGCGGGTTAGCGATTCGACCCTCCTGCCCAAGGC
TTTTATGGAAATTGCCGTTAACATGGCTCGCGTGTCCCATTGTACCTACC
AGTACGGCGACGGTTTGGGCCGTCCGGATTACGCAACCGAGAACCGCATC
AAGCTCCTTCTGATCGACCCGTTCCCGATCAACCAGCTCATGTATGTCTA
Atctaga

SEQ ID NO:5 IspS Amino Acid Sequence for expression in yeast or Neurospora crassa A starting methionine encoded by the cDNA is not shown in the amino acid sequence.

ICATSSQFTQITEHNSRRSANYQPNLWNFEFLQSLENDLKVEKLEEKATK
LEEEVRCMINRVDTQPLSLLELIDDVQRLGLTYKFEKDIIKALENIVLLD
ENKKNKSDLHATALSFRLLRQHGFEVSQDVFERFKDKEGGFSGELKGDVQ
GLLSLYEASYLGFEGENLLEEARTFSITHLKNNLKEGINTKVAEQVSHAL
ELPYHQRLHRLEARWFLDKYEPKEPHHQLLLELAKLDFNMVQTLHQKELQ
DLSRWWTEMGLASKLDFVRDRLMEVYFWALGMAPDPQFGECRKAVTKMFG
LVTIIDDVYDVYGTLDELQLFTDAVERWDVNAINTLPDYMKLCFLALYNT
VNDTSYSILKEKGHNNLSYLTKSWRELCKAFLQEAKWSNNKIIPAFSKYL
ENASVSSSGVALLAPSYFSVCQQQEDISDHALRSLTDFHGLVRSSCVIFR
LCNDLATSAAELERGETTNSIISYMHENDGTSEEQAREELRKLIDAEWKK
MNRERVSDSTLLPKAFMEIAVNMARVSHCTYQYGDGLGRPDYATENRIKL
LLIDPFPINQLMYV

SEQ ID NO:6 Populus alba cDNA for isoprene synthase, Accession No. AB198180

| | |
|---|---|
| 1 | atggcaactg aattattgtg cttgcaccgt ccaatctcac tgacacacaa acttttcaga |
| 61 | aatcccttac ctaaagtcat ccaggccact cccttaactt tgaaactcag atgttctgta |
| 121 | agcacagaaa acgtcagctt cacagaaaca gaaacagaag ccagacggtc tgccaattat |
| 181 | gaaccaaata gctgggatta tgattatttg ctgtcttcag acactgacga atcgattgag |
| 241 | gtatacaaag acaaggccaa aaagctggag gctgaggtga gaagagagat taacaatgaa |
| 301 | aaggcagagt ttttgactct gcttgaactg atagataatg tccaaaggtt aggattgggt |
| 361 | taccggttcg agagtgacat aaggggagcc cttgatagat ttgtttcttc aggaggattt |
| 421 | gatgctgtta caaaaactag ccttcatggt actgctctta gcttcaggct tctcagacag |
| 481 | catggttttg aggtctctca agaagcgttc agtggattca aggatcaaaa tggcaatttc |
| 541 | ttggaaaacc ttaaggagga catcaaggca atactaagcc tatatgaagc ttcatttctt |
| 601 | gcattagaag gagaaaatat cttggatgag gccaaggtgt ttgcaatatc acatctaaaa |
| 661 | gagctcagcg aagaaaagat tggaaaagag ctggccgaac aggtgaatca tgcattggag |
| 721 | cttccattgc atcgcaggac gcaaagacta gaagctgttt ggagcattga agcataccgt |
| 781 | aaaaaggaag atgcaaatca agtactgcta gaacttgcta tattggacta caacatgatt |
| 841 | caatcagtat accaaagaga tcttcgcgag acatcaaggt ggtggaggcg agtgggtctt |
| 901 | gcaacaaagt tgcattttgc tagagacagg ttaattgaaa gcttttactg ggcagttgga |
| 961 | gttgcgttcg agcctcaata cagtgattgc cgtaattcag tagcaaaaat gttttcattt |
| 1021 | gtaacaatca ttgatgatat ctatgatgtt tatggtactc tggacgagtt ggagctattt |
| 1081 | acagatgctg ttgagagatg ggatgttaat gccatcaatg atcttccgga ttatatgaag |
| 1141 | ctctgcttcc tagctctcta caacactatc aatgagatag cttatgacaa tctgaaggac |
| 1201 | aagggggaaa acattcttcc atacctaaca aaagcgtggg cagatttatg caatgcattc |
| 1261 | ctacaagaag caaaatggtt gtacaataag tccacaccaa catttgatga ctatttcgga |
| 1321 | aatgcatgga aatcatcctc agggcctctt caactagttt ttgcctactt tgccgtggtt |
| 1381 | caaaacatca agaaagagga aattgaaaac ttacaaaagt atcatgatac catcagtagg |
| 1441 | ccttcccaca tctttcgtct ttgcaacgac ctggcttcag catcggctga gatagcgaga |
| 1501 | ggtgaaacag cgaattctgt atcatgctac atgcgtacaa aaggcatttc tgaggagctt |
| 1561 | gctactgaat ccgtaatgaa cttgatcgac gaaacctgga aaaagatgaa caaagaaaag |
| 1621 | cttggtggct ctttgtttgc aaaacctttt gtcgaaacag ctattaacct tgcacggcaa |
| 1681 | tcccattgca cttatcataa cggagatgcg catacttcac cagacgagct aactaggaaa |
| 1741 | cgtgtcctgt cagtaatcac agagcctatt ctaccctttg agagataa |

SEQ ID NO:7 Populus alba polypeptide sequence for isoprene synthase (from Accession No. AB198180). The underlined portion of the protein denotes a chloroplast transit peptide.

MATELLCLHRPISLTHKLFRNPLPKVIQATPLTLKLRCSVSTENVSFTET
ETEARRSANYEPNSWDYDYLLSSDTDESIEVYKDKAKKLEAEVRREINNE
KAEFLTLLELIDNVQRLGLGYRFESDIRGALDRFVSSGGFDAVTKTSLHG
TALSFRLLRQHGFEVSQEAFSGFKDQNGNFLENLKED1KAILSLYEASFL
ALEGENILDEAKVFAISHLKELSEEKIGKELAEQVNHALELPLHRRTQRL
EAVWSIEAYRKKEDANQVLLELAILDYNMIQSVYQRDLRETSRWWRRVGL
ATKLHFARDRLIESFYWAVGVAFEPQYSDCRNSVAKMFSFVTIIDDIYDV
YGTLDELELFTDAVERWDVNAINDLPDYMKLCFLALYNTINEIAYDNLKD
KGENILPYLTKAWADLCNAFLQEAKWLYNKSTPTFDDYFGNAWKSSSGPL
QLVFAYFAVVQNIKKEEIENLQKYHDTISRPSHIFRLCNDLASASAEIAR
GETANSVSCYMRTKGISEELATESVMNLIDETWKKMNKEKLGGSLFAKPF
VETAINLARQSHCTYHNGDAHTSPDELTRKRVLSVITEPILPFER

SEQ ID NOs. 8-11: Alternative versions of codon-optimized IspS sequences for expression in yeast that encode an IspS protein of SEQ ID NO:5 Sc-k-IspS; Version 1

GCGATCGCACACACATAAATAAACAAAATGATCTGTGCAACTTCTTCCCA
ATTCACCCAAATAACCGAACATAATTCCCGTAGAAGTGCAAATTACCAAC
CAAATTTGTGGAATTTTGAATTTCTTCAATCCTTGGAGAACGATTTGAAA
GTGGAAAAGTTGGAGGAAAAAGCTACCAAGCTTGAAGAAGAAGTGAGATG
TATGATAAACAGAGTGGATACCCAGCCATTATCTTTGTTAGAACTAATCG
ATGACGTTCAAAGACTAGGTCTAACATATAAGTTTGAGAAGGATATTATT
AAAGCATTGGAAAATATAGTTTTACTTGATGAAAACAAGAAGAATAAGAG
TGATTTACACGCTACAGCATTGTCTTTTAGACTTTTGAGGCAGCATGGAT
TTGAAGTCTCACAAGACGTTTTTGAAAGATTCAAGGACAAAGAAGGAGGG
TTTTCCGGAGAATTAAAAGGTGATGTCCAAGGTTTATTGAGTTTGTACGA
AGCTTCTTATTTGGGTTTTGAAGGTGAAAACTTATTAGAAGAAGCTAGAA
CATTCTCCATCACTCACTTAAAGAATAATCTTAAAGAGGGAATTAATACC
AAAGTTGCCGAACAAGTCTCACATGCACTAGAATTACCATATCATCAAAG
ATTGCACCGTTTAGAAGCTAGATGGTTCTTAGACAAGTATGAACCAAAGG
AACCTCATCACCAATTGTTGTTGGAATTAGCAAAGCTAGATTTCAACATG
GTCCAAACATTGCACCAAAAGGAACTTCAGGACCTTTCTAGGTGGTGGAC
AGAAATGGGATTAGCCAGTAAGTTGGACTTTGTCAGAGATAGGTTAATGG
AAGTCTATTTTTGGGCACTAGGAATGGCACCTGATCCACAGTTTGGGGAG
TGCAGAAAAGCTGTAACAAAAATGTTCGGCTTAGTAACAATTATAGATGA
TGTGTACGACGTGTATGGCACACTTGACGAACTTCAACTTTTTACTGATG
CCGTTGAGAGGTGGGATGTGAATGCTATTAATACATTACCCGATTATATG
AAGTTGTGCTTCTTAGCCTTATATAATACTGTCAACGATACTTCCTATAG
TATCTTAAAAGAGAAGGGTCACAATAATCTTAGTTATTTGACTAAAAGTT
GGCGTGAACTTTGTAAAGCCTTTCTTCAAGAAGCTAAGTGGTCTAATAAT
AAGATTATTCCTGCCTTTTCCAAATATCTTGAAAACGCCTCTGTATCCTC
TTCCGGTGTTGCATTGTTAGCTCCTTCTTATTTCTCCGTTTGCCAACAAC
AGGAAGATATTTCAGATCACGCCTTGAGATCTCTTACGGATTTTCATGGT
TTAGTGCGTAGTAGTTGCGTTATTTTTCGTTTGTGCAATGACTTAGCTAC
TTCCGCAGCTGAATTGGAAAGAGGTGAAACTACCAATTCAATTATCTCTT
ACATGCATGAAAATGATGGCACATCCGAAGAACAGGCTCGTGAAGAGTTA
AGGAAGTTGATCGACGCAGAGTGGAAGAAAATGAATAGGGAGAGAGTCAG
TGATTCTACATTGTTACCAAAGGCATTTATGGAAATAGCAGTAAACATGG
CAAGAGTTAGTCATTGTACATATCAATACGGTGATGGCTTGGGGCGTCCA
GATTACGCTACAGAGAATAGAATTAAGTTATTGTTGATTGATCCTTTTCC
GATTAACCAATTAATGTACGTGTAAGGCCGGCCATCCTTGACAGGCGCGC
C

Annotations for underlined and italicized sequences in SEQ ID NOs. 8-11:

GCGATCGC=SgfI restriction site

ACACACATAAATAAACAAA=Portion of the GPD promoter region missing from the ATCC plasmid.

GGCCGGCC=FseI

ATCCTTGACA=Spacer

GGCGCGCC=AscI restriction site

Sc-k-IspS; Version 2

GCGATCGCACACACATAAATAAACAAAATGATATGTGCGACTTCAAGTCA
ATTCACTCAGATTACCGAGCATAACTCGAGGAGATCTGCGAATTACCAAC
CCAATTTGTGGAATTTCGAGTTTCTTCAGTCTCTGGAAAATGACTTAAAG
GTTGAAAAACTAGAAGAGAAAGCTACTAAGTTAGAAGAAGAAGTCAGGTG
CATGATAAACAGGGTGGATACTCAACCGTTGAGCTTGTTAGAACTAATAG
ATGATGTTCAAAGATTAGGTCTAACGTACAAATTCGAAAAGGACATAATC
AAAGCTCTAGAAAACATAGTCTTGCTTGACGAAAACAAAAAGAACAAAAG
TGACTTACATGCTACCGCCTTATCGTTTAGATTGCTGCGTCAACATGGAT
TTGAAGTAAGTCAAGACGTTTTCGAGAGATTCAAAGATAAAGAAGGCGGT
TTTTCTGGTGAATTGAAGGGAGATGTGCAAGGGCTGTTGTCCTTGTATGA
GGCTTCCTATCTAGGCTTCGAAGGCGAAAATCTACTGGAAGAGGCCAGAA
CATTCAGCATTACACATTTGAAGAATAACTTGAAAGAGGGGATTAACACT
AAGGTTGCTGAACAGGTATCCCACGCACTAGAATTACCATATCACCAAAG
ATTACACCGTTTAGAAGCGAGATGGTTTTTGGACAAGTATGAACCAAAAG
AACCGCACCATCAGCTTCTTTTAGAGTTGGCAAAACTAGATTTCAATATG
GTTCAGACTTTACATCAAAAAGAATTACAAGATTTGAGCAGATGGTGGAC
CGAAATGGGACTTGCCTCCAAGTTAGACTTTGTGCGTGACCGTCTTATGG
AAGTTTACTTCTGGGCCTTAGGGATGGCACCAGATCCTCAGTTTGGTGAA
TGTAGGAAAGCTGTCACTAAGATGTTTGGACTGGTTACAATCATTGATGA
TGTTTACGATGTTTATGGAACCTTAGACGAATTACAATTGTTCACCGATG
CAGTAGAGAGATGGGATGTGAATGCCATTAACACACTGCCAGACTACATG
AAGTTGTGTTTCTTAGCTTTGTACAACACAGTTAATGATACTTCATATTC
TATTCTTAAAGAGAAAGGTCATAACAACTTATCCTATTTGACGAAATCAT
GGAGAGAGCTATGTAAAGCTTTCTTACAAGAAGCCAAGTGGTCTAACAAC
AAAATCATTCCCGCTTTTTCGAAGTACTTGGAGAATGCATCTGTTAGTTC
ATCAGGTGTCGCGTTACTAGCGCCTAGTTACTTTTCTGTCTGCCAACAGC
AAGAAGATATCAGCGATCATGCATTGAGGTCTTTGACCGATTTTCATGGC
TTAGTTAGAAGCAGTTGCGTCATTTTCAGGCTATGTAATGACTTGGCTAC
ATCTGCTGCTGAACTTGAGCGTGGTGAGACAACTAATTCCATCATTTCGT
ATATGCACGAAAACGACGGTACTTCTGAAGAACAGGCCAGAGAAGAGTTA
AGAAAGTTAATCGACGCAGAATGGAAGAAGATGAATAGAGAGCGTGTGTC
AGATTCTACGTTACTGCCTAAAGCCTTTATGGAAATAGCAGTGAATATGG
CAAGAGTATCACATTGTACATATCAATACGGTGACGGTCTTGGTAGACCT
GATTATGCAACGGAAAACAGAATCAAATTGCTTCTTATTGATCCATTTCC
TATCAATCAACTAATGTACGTATAAGGCCGGCCATCCTTGACAGGCGCGC
C

Sc-k-IspS; Version 3

GCGATCGCACACACATAAATAAACAAAATGATATGTGCTACTTCAAGTCA
ATTCACTCAGATTACCGAGCATAACTCGAGGAGATCTGCCAATTACCAAC
CCAATTTGTGGAATTTCGAGTTTCTTCAGTCTCTGGAAAATGACTTAAAG
GTTGAAAAACTAGAAGAGAAAGCTACTAAGTTAGAAGAAGAAGTCAGGTG
CATGATAAACAGGGTGGATACTCAACCGTTGAGCTTGTTAGAACTAATAG
ATGATGTTCAAAGATTAGGTCTAACGTACAAATTCGAAAAGGACATAATC
AAAGCTCTAGAAAACATAGTCTTGCTTGACGAAAACAAAAAGAACAAAAG
TGACTTACATGCTACCGCCTTATCGTTTAGATTGCTGCGTCAACATGGAT
TTGAAGTAAGTCAAGACGTTTTCGAGAGATTCAAAGATAAAGAAGGCGGT
TTTTCTGGTGAATTGAAGGGAGATGTGCAAGGGCTGTTGTCCTTGTATGA
GGCTTCCTATCTAGGCTTCGAAGGCGAAAATCTACTGGAAGAGGCCAGAA
CATTCAGCATTACACATTTGAAGAATAACTTGAAAGAGGGGATTAACACT
AAGGTTGCTGAACAGGTATCCCACGCACTAGAATTACCATATCACCAAAG
ATTACACCGTTTAGAAGCTAGATGGTTTTTGGACAAGTATGAACCAAAAG
AACCGCACCATCAGCTTCTTTTAGAGTTGGCAAAACTAGATTTCAATATG
GTTCAGACTTTACATCAAAAAGAATTACAAGATTTGAGCAGATGGTGGAC
CGAAATGGGACTTGCCTCCAAGTTAGACTTTGTGCGTGACCGTCTTATGG
AAGTTTACTTCTGGGCCTTAGGGATGGCACCAGATCCTCAGTTTGGTGAA
TGTAGGAAAGCTGTCACTAAGATGTTTGGACTGGTTACAATCATTGATGA
TGTTTACGATGTTTATGGAACCTTAGACGAATTACAATTGTTCACCGATG
CAGTAGAGAGATGGGATGTGAATGCCATTAACACACTGCCAGACTACATG
AAGTTGTGTTTCTTAGCTTTGTACAACACAGTTAATGATACTTCATATTC
TATTCTTAAAGAGAAAGGTCATAACAACTTATCCTATTTGACGAAATCAT
GGAGAGAGCTATGTAAAGCTTTCTTACAAGAAGCCAAGTGGTCTAACAAC
AAAATCATTCCCGCTTTTTCGAAGTACTTGGAGAATGCATCTGTTAGTTC
ATCAGGTGTCGCGTTACTAGCGCCTAGTTACTTTTCTGTCTGCCAACAGC
AAGAAGATATCAGCGATCATGCATTGAGGTCTTTGACCGATTTTCATGGC
TTAGTTAGAAGCAGTTGCGTCATTTTCAGGCTATGTAATGACTTGGCTAC
ATCTGCTGCTGAACTTGAGCGTGGTGAGACAACTAATTCCATCATTTCGT
ATATGCACGAAAACGACGGTACTTCTGAAGAACAGGCCAGAGAAGAGTTA
AGAAAGTTAATCGACGCAGAATGGAAGAAGATGAATAGAGAGCGTGTGTC
AGATTCTACGTTACTGCCTAAAGCCTTTATGGAAATAGCAGTGAATATGG
CAAGAGTATCACATTGTACATATCAATACGGTGACGGTCTTGGTAGACCT
GATTATGCAACGGAAAACAGAATCAAATTGCTTCTTATTGATCCATTTCC
TATCAATCAACTAATGTACGTATAAGGCCGGCCATCCTTGACAGGCGCGC
C

Sc-k-IspS; Version 4

GCGATCGCACACACATAAATAAACAAAATGATTTGTGCTACATCCTCACA
ATTCACTCAGATAACTGAACATAACTCTAGAAGATCAGCCAATTACCAAC
CCAATTTGTGGAATTTCGAATTCTTACAGAGTCTTGAGAATGATCTAAAA
GTAGAAAAGTTAGAGGAAAAAGCTACGAAACTTGAAGAAGAGGTTAGATG
CATGATTAACAGAGTAGATACACAGCCTCTATCATTACTAGAACTTATAG
ATGACGTCCAAAGACTAGGTCTTACTTACAAGTTTGAGAAAGACATAATC
AAAGCCTTAGAAAACATTGTTCTTTTAGATGAAAACAAGAAAAACAAGTC
TGATTTGCACGCTACGGCATTGAGTTTTAGGTTGTTGAGACAGCATGGCT
TTGAAGTGTCACAAGATGTTTTTGAAAGATTCAAAGATAAAGAAGGTGGC
TTTAGTGGGGAGTTGAAAGGTGACGTCCAGGGCTTACTTTCTTTGTACGA
AGCCTCTTATCTTGGTTTCGAAGGAGAAAATCTACTTGAAGAGGCCAGAA
CATTTTCAATTACCCATTTGAAGAATAACTTGAAAGAGGGTATCAATACA
AAGGTTGCAGAGCAAGTATCTCATGCTTTAGAGCTACCTTATCATCAAAG
ATTGCATCGTTTGGAAGCTAGATGGTTTCTAGATAAGTACGAACCAAAAG
AACCACACCATCAACTTTTGTTAGAATTAGCCAAGTTGGATTTCAACATG
GTCCAGACACTTCACCAAAAAGAGTTACAAGATTTGTCAAGATGGTGGAC
CGAAATGGGTTTAGCCAGTAAACTAGACTTCGTACGTGATAGGTTAATGG
AGGTATACTTTTGGGCCTTAGGGATGGCTCCTGACCCACAATTTGGTGAA
TGTAGAAAAGCTGTTACCAAAATGTTTGGATTAGTAACAATCATTGACGA
CGTTTATGACGTTTACGGTACTCTTGATGAATTACAATTGTTTACCGACG
CTGTTGAACGTTGGGACGTTAATGCTATCAACACTCTTCCAGACTATATG
AAACTTTGCTTTTTGGCATTGTATAACACAGTTAATGATACGTCTTACTC
TATTCTTAAAGAAAAAGGTCACAATAACTTATCTTACTTGACTAAGTCTT
GGAGGGAATTGTGCAAAGCCTTCTTACAAGAGGCAAAGTGGTCAAACAAT
AAGATCATTCCGGCATTCTCCAAGTATTTGGAAAACGCTAGTGTGTCCTC
ATCAGGAGTGGCTTTGTTGGCACCTAGTTACTTCTCTGTCTGTCAACAAC
AAGAGGATATCAGTGATCATGCCCTAAGGTCATTAACAGATTTTCATGGT
TTAGTCAGATCTTCATGTGTCATATTCAGATTGTGTAATGACTTAGCAAC
AAGTGCAGCTGAATTAGAACGTGGCGAAACCACTAATTCCATTATCTCCT
ATATGCACGAGAATGATGGGACTTCAGAAGAACAGGCAAGGGAGGAGTTG
CGTAAACTAATCGATGCTGAATGGAAGAAAATGAATAGAGAACGTGTGTC
TGATTCCACCCTATTACCAAAGGCTTTCATGGAAATTGCCGTGAATATGG
CAAGGGTTTCCCATTGTACGTATCAATATGGAGATGGATTAGGTAGGCCC
GACTATGCTACAGAGAATAGGATAAAGCTACTATTGATAGATCCATTTCC
GATTAACCAGCTTATGTATGTATAAGGCCGGCCATCCTTGACAGGCGCGC
C

What is claimed is:

1. A method of producing isoprene hydrocarbons in a Ascomycota microorganism selected from the group consisting of yeast or filamentous fungi, the method comprising:
introducing an expression cassette that comprises a nucleic acid encoding isoprene synthase into the microorganism; and
culturing the microorganism under conditions in which the nucleic acid encoding isoprene synthase is expressed.

2. The method of claim 1, wherein the microorganism is a yeast.

3. The method of claim 2, wherein the yeast is Saccharomyces cerevisiae.

4. The method of claim 1, wherein the microorganism is a filamentous fungi.

5. The method of claim 4, wherein the filamentous fungi is Neurospora crassa or Aspergillus fumigatus.

6. The method of claim 1, wherein the nucleic acid encodes an isoprene synthase that comprises the amino acid sequence set forth in SEQ ID NO:5.

7. The method of claim 1, wherein the microorganism overexpresses HMG-CoA synthase and/or HMG-CoA reductase.

8. A microorganism selected from the group consisting of a yeast cell or a filamentous fungi cell, wherein the microorganism comprises a heterologous nucleic acid that encodes isoprene synthase and is operably linked to a promoter, wherein the organism exhibits increased expression of isoprene syntase.

9. The microorganism of claim 8, wherein the microorganism is a yeast cell.

10. The microorganism of claim 9, wherein the yeast is Saccharomyces cerevisiae.

11. The microorganism of claim 8, wherein the microorganism is a filamentous fungus.

12. The microorganism of claim 11, wherein the filamentous fungus is Neurospora crassa or Aspergillus fumigatus.

13. The microorganism of claim 8, wherein the heterologous nucleic acid encodes an isoprene synthase that comprises the amino acid sequence set forth in SEQ ID NO:5.

14. A method of producing isoprene hydrocarbons in a Ascomycota microorganism that comprises a heterologous nucleic acid that encodes isoprene synthase, the method comprising:
mass-culturing the microorganism in an enclosed bioreactor under conditions in which the isoprene synthase gene is expressed; and
harvesting volatile isoprene hydrocarbons produced by the microorganism.

15. The method of claim 14, wherein the microorganism is a yeast.

16. The method of claim 15, wherein the yeast is Saccharomyces cerevisiae.

17. The method of claim 14 wherein the microorganism is a filamentous fungi.

18. The method of claim 17, wherein the filamentous fungi is Neurospora crassa or Aspergillus fumigatus.

19. The method of claim 14, wherein the heterologous nucleic acid encodes an isoprene synthase that comprises the amino acid sequence set forth in SEQ ID NO:5.
